(19) United States
(12) Patent Application Publication
(10) Pub. No.: US 2022/0387350 A1
(43) Pub. Date: Dec. 8, 2022

(54) CANNABIDIVARIN PREPARATIONS FOR USE AS A MEDICAMENT

(71) Applicant: GW Research Limited, Cambridge (GB)

(72) Inventors: Geoffrey GUY, Cambridge (GB); Volker KNAPPERTZ, Cambridge (GB); Benjamin WHALLEY, Cambridge (GB); Marie WOOLLEY-ROBERTS, Cambridge (GB)

(21) Appl. No.: 17/770,436

(22) PCT Filed: Oct. 23, 2020
(86) PCT No.: PCT/GB2020/052694

§ 371 Date: Apr. 20, 2022

(30) Foreign Application Priority Data
Oct. 25, 2019 (GB) 1915516.7

(51) Int. Cl. A61K 31/05; A61K 36/185; A61P 25/18

(52) U.S. Cl. CPC A61K 31/05; A61K 36/185; A61P 25/18; A61K 2236/37; A61K 2236/53

(57) ABSTRACT

Cannabidivarin (CBDV) is a cannabinoid designated chemically as 2-[(1R,6R)-3-Methyl-6-(1-methylethenyl)-2-cyclohexen-1-yl]-5-propyl-1,3-benzenediol. Its empirical formula is C19H26O2 and its molecular weight is 286.4 g/mol. CBDV is a cannabinoid that naturally occurs in the Cannabis sativa L. plant. CBDV is an off-white to pale yellow crystalline solid which is insoluble in water and soluble in organic solvents. The present invention encompasses the surprising recognition that certain CBDV preparations which are prepared from a botanical origin are more effective in treating diseases or disorders than preparations of CBDV which are synthetic or purified to the extent no other impurities in the form of other cannabinoids are present. Prior CBDV compositions have been prepared such that no psychoactive components, e.g., tetrahydrocannabinol (THC), remain in the final CBDV preparation. Surprisingly, the absence of such minor impurities reduces the efficacy of CBDV treatment. Such CBDV preparations are characterized by chemical components and/or functional properties that distinguish them from prior CBDV compositions. One or more components of the preparations described herein provide an unexpectedly synergistic effect when utilized in combination.

### FIELD OF THE INVENTION

Cannabidivarin (CBDV) is a cannabinoid designated chemically as 2-[(1R,6R)-3-Methyl-6-(1-methylethenyl)-2-cyclohexen-1-yl]-5-propyl-1,3-benzenediol. Its empirical formula is C₁₉H₂₆O₂ and its molecular weight is 286.4 g/mol. CBDV is a cannabinoid that naturally occurs in the Cannabis sativa L. plant. CBDV is an off-white to pale yellow crystalline solid which is insoluble in water and soluble in organic solvents.

The present invention encompasses the surprising recognition that certain CBDV preparations which are prepared from a botanical origin are more effective in treating diseases or disorders than preparations of CBDV which are synthetic or purified to the extent no other impurities in the form of other cannabinoids are present.

Prior CBDV compositions have been prepared such that no psychoactive components, e.g., tetrahydrocannabinol (THC), remain in the final CBDV preparation. Surprisingly, the absence of such minor impurities reduces the efficacy of CBDV treatment.

Such CBDV preparations are characterized by chemical components and/or functional properties that distinguish them from prior CBDV compositions. One or more components of the preparations described herein provide an unexpectedly synergistic effect when utilized in combination.

### BACKGROUND TO THE INVENTION

Cannabinoids are natural and synthetic compounds structurally or pharmacologically related to the constituents of the cannabis plant or to the endogenous agonists (endocannabinoids) of the cannabinoid receptors CB1 or CB2. The only way in nature in which these compounds are produced is by the cannabis plant. Cannabis is a genus of flowering plants in the family Cannabaceae, comprising the species Cannabis sativa, Cannabis indica, and Cannabis ruderalis (sometimes considered as part of Cannabis sativa).

Cannabis plants comprise a highly complex mixture of compounds. At least 568 unique molecules have been identified. Among these compounds are cannabinoids, terpenoids, sugars, fatty acids, flavonoids, other hydrocarbons, nitrogenous compounds, and amino acids.

Cannabinoids exert their physiological effects through a variety of receptors including, but not limited to, adrenergic receptors, cannabinoid receptors (CB1 and CB2), GPR55, GPR3, or GPR5. The principle cannabinoids present in cannabis plants are cannabinoid acids tetrahydrocannabinolic acid (THCA) and cannabidiolic acid (CBDA) with small amounts of their respective neutral (decarboxylated) cannabinoids. In addition, cannabis may contain lower levels of other minor cannabinoids.

Crude extracts from cannabis plants containing CBDV have been used by patients suffering from diseases and disorders. However, such crude products are unsuitable for use in pharmaceutical formulations. Those seeking to prepare more consistent CBDV preparations for use in treating diseases or disorders have made a concerted effort to either prepare CBDV synthetically or attempt to remove all compounds other than CBDV, particularly psychoactive compounds such as THC, from plant derived cannabinoids.

The present invention encompasses the surprising discovery that particular preparations comprising CBDV have an improved therapeutic efficacy in comparison to synthetic preparations of CBDV which comprise no minor cannabinoid impurities and crude extracts which have higher levels of the minor cannabinoid impurities.

As stated, cannabinoids are a class of compounds which may be derived naturally from the cannabis plant or produced synthetically via chemical synthesis.

More than 100 different cannabinoids produced by cannabis have been identified as described in Handbook of Cannabis, Roger Pertwee, Chapter 1, pages 3 to 15. These cannabinoids can be split into different groups as follows: phytocannabinoids; endocannabinoids and synthetic cannabinoids (which may be novel cannabinoids or synthetically produced versions of phytocannabinoids or endocannabinoids).

Phytocannabinoids are cannabinoids that originate from nature and can be found in the cannabis plant. Phytocannabinoids can be isolated from plants to produce a highly purified extract. Phytocannabinoids may be obtained as either the neutral (decarboxylated form) or the carboxylic acid form depending on the method used to extract the cannabinoids from plant material. For example, it is known that heating the carboxylic acid form will cause most of the carboxylic acid form to decarboxylate into the neutral form. Phytocannabinoids can only be produced from plants, however versions of phytocannabinoids may be produced synthetically via chemical synthesis.

Endocannabinoids are endogenous lipid-based retrograde neurotransmitters that bind to cannabinoid receptors, and cannabinoid receptor proteins that are expressed throughout the mammalian central nervous system (including the brain) and peripheral nervous system. The endocannabinoid system is involved in regulating a variety of physiological and cognitive processes including fertility, pregnancy, during pre- and postnatal development, appetite, pain-sensation, mood, and memory, and in mediating the pharmacological effects of cannabis.

Synthetic cannabinoids are compounds that have a cannabinoid-like structure and are manufactured using chemical means rather than by the plant.

Certain cannabinoids are described in more detail below.

Of the over 100 natural cannabinoids identified in Cannabis sativa, seven have been classified as CBD-type compounds, these cannabinoids have the same absolute configuration as CBD. These are: cannabidiol (CBD), Cannabidiolic acid (CBDA), Cannabidivarin (CBDV), Cannabidivarin acid (CBDVA), Cannabidiol-C1 (CBD-C1), Cannabidiol-C4 (CBD-C4) and Cannabidiol monomethyl ether (CBDM).

Cannabidiol (CBD) is a major cannabinoid constituent of Cannabis species, such as the hemp plant (Cannabis sativa). Unlike other cannabinoids, such as THC, cannabidiol does not bind to CB1 or CB2, or its binding to the receptors is negligible in terms of inducing a pharmacological effect. Thus, cannabidiol does not cause the central or peripheral nervous system effects mediated by the CB1 or CB2 receptors. CBD has little or no psychotropic (cannabimimetic) activity and its molecular structure and properties are substantially different from those of other cannabinoids.

Cannabidivarin (CBDV) is a homolog of CBD, with the sidechain shortened by two methylene bridges. CBDV is a non-psychoactive cannabinoid and has been shown to have anti-convulsant activity in a mouse model of epilepsy.

Cannabidiol administration has been the subject of research in an attempt to provide an alternative treatment for various diseases and disorders which may respond to such treatment.

Cannabidiolic acid (CBDA) is the main form in which CBD exists in the cannabis plant. It is converted into CBD after decarboxylation.

Cannabidiol-C1 (CBD-C1) also known as cannabidiorcol is a homolog of CBD, with the side-chain shortened by four methylene bridges. CBD-C1 occurs naturally in plants producing CBD in minor quantities.

Cannabidiol-C4 (CBD-C4) also known as nor-cannabidiol is a homolog of CBD, with the side-chain shortened by one methylene bridge. CBD-C4 occurs naturally in plants producing CBD in minor quantities.

Tetrahydrocannabinol (THC) is the principal psychoactive constituent of cannabis. THC is a partial agonist at the CB1 and CB2 receptors. Synthetic THC or dronabinol is approved for the treatment of loss of appetite in AIDS patients and nausea and vomiting caused by cancer chemotherapy. The cannabimimetic side effects caused by THC include feeling high, nausea, vomiting, anxiety, depression and weakness.

Tetrahydrocannabivarin (THCV) is a homolog of THC, with the sidechain shortened by two methylene bridges. THCV is a non-psychoactive cannabinoid and has been shown to produce appetite suppression in an animal model of obesity.

The present invention demonstrates an increased efficacy of a botanically derived purified CBDV preparation which comprises minor amounts of the cannabinoids CBD-C1, CBD, CBD-C4, CBDVA, THCV and THC over a synthetic CBDV which does not comprise minor amounts of cannabinoids. These data are particularly surprising particularly given the fact that the concentration of CBDV within the botanically derived purified CBDV preparation and the synthetic preparation were the same. The invention further discloses the difference in the physicochemical properties of a botanically derived purified CBDV and a synthetic CBDV.

### BRIEF SUMMARY OF THE DISCLOSURE

In accordance with a first aspect of the present invention there is provided a cannabidivarin (CBDV) preparation characterized in that it comprises greater than or equal to 95% (w/w) CBDV and less than or equal to 5% (w/w) other cannabinoids, wherein the less than or equal to 5% (w/w) other cannabinoids comprise the cannabinoids tetrahydrocannabinol (THC); tetrahydrocannabivarin (THCV); cannabidiol-C1 (CBD-C1); cannabidiol (CBD); cannabidivarin acid (CBDV) and cannabidiol-C4 (CBD-C4).

In accordance with a second aspect of the present invention there is provided a cannabidivarin (CBDV) preparation characterized in that it comprises greater than or equal to 95% (w/w) CBDV and less than or equal to 5% (w/w) other cannabinoids, wherein the less than or equal to 5% (w/w) other cannabinoids comprise the cannabinoids tetrahydrocannabinol (THC); tetrahydrocannabivarin (THCV); cannabidiol-C1 (CBD-C1); cannabidiol (CBD); cannabidivarin acid (CBDV) and cannabidiol-C4 (CBD-C4) for use as a medicament.

In accordance with a third aspect of the present invention there is provided a cannabidivarin (CBDV) preparation characterized in that it comprises greater than or equal to 95% (w/w) CBDV and less than or equal to 5% (w/w) other cannabinoids, wherein the less than or equal to 5% (w/w) other cannabinoids comprise the cannabinoids tetrahydrocannabinol (THC); tetrahydrocannabivarin (THCV); cannabidiol-C1 (CBD-C1); cannabidiol (CBD); cannabidivarin acid (CBDV) and cannabidiol-C4 (CBD-C4) for use in the treatment of neurodevelopmental diseases and conditions.

In accordance with a fourth aspect of the present invention there is provided a cannabidivarin (CBDV) preparation characterized in that it comprises greater than or equal to 95% (w/w) CBDV and less than or equal to 5% (w/w) other cannabinoids, wherein the less than or equal to 5% (w/w) other cannabinoids comprise the cannabinoids tetrahydrocannabinol (THC); tetrahydrocannabivarin (THCV); cannabidiol-C1 (CBD-C1); cannabidiol (CBD); cannabidivarin acid (CBDV) and cannabidiol-C4 (CBD-C4) for use in the treatment of epilepsy.

In accordance with a fifth aspect of the present invention there is provided a cannabidivarin (CBDV) preparation characterized in that it comprises greater than or equal to 95% (w/w) CBDV and less than or equal to 5% (w/w) other cannabinoids, wherein the less than or equal to 5% (w/w) other cannabinoids comprise the cannabinoids tetrahydrocannabinol (THC); tetrahydrocannabivarin (THCV); cannabidiol-C1 (CBD-C1); cannabidiol (CBD); cannabidivarin acid (CBDV) and cannabidiol-C4 (CBD-C4) for use in the treatment of schizophrenia.

Preferably the preparation comprises not more than 1.5% (w/w) THC based on total amount of cannabinoid in the preparation. More preferably the preparation comprises about 0.01% to about 0.1% (w/w) THC based on total amount of cannabinoid in the preparation. More preferably still the preparation comprises about 0.02% to about 0.05% (w/w) THC based on total amount of cannabinoid in the preparation.

Preferably the preparation comprises about 0.001% to about 0.01% (w/w) THCV based on total amount of cannabinoid in the preparation.

Preferably the preparation comprises about 0.1% to about 0.25% (w/w) CBD-C1 based on total amount of cannabinoid in the preparation.

Preferably the preparation comprises about 1% to about 5% (w/w) CBD based on total amount of cannabinoid in the preparation.

Preferably the preparation comprises about 0.1% to about 0.25% (w/w) CBDVA based on total amount of cannabinoid in the preparation.

Preferably the preparation comprises about 0.05% to about 0.5% (w/w) CBD-C4 based on total amount of cannabinoid in the preparation.

In one embodiment at least a portion of at least one of the cannabinoids present in the CBDV preparation is isolated from cannabis plant material.

In a further embodiment of the invention substantially all of at least one of the cannabinoids present in the CBDV preparation is isolated from cannabis plant material.

In a further embodiment of the invention substantially all of the cannabinoids present in the CBDV preparation are isolated from cannabis plant material.

In a further embodiment of the invention at least a portion of at least one of the cannabinoids present in the CBDV preparation is prepared synthetically.

In a further embodiment of the invention substantially all of at least one of the cannabinoids present in the CBDV preparation is prepared synthetically.

Preferably substantially all of the cannabinoids present in the CBDV preparation are prepared synthetically.

In a further embodiment of the invention the neurodegenerative disease or disorder is Alzheimer's disease; Parkinson's disease; essential tremor; amyotrophic lateral sclerosis (ALS); Huntington's disease; Friedreich's ataxia; multiple sclerosis; frontotemporal dementia; prion disease; Lewy body dementia; progressive supranuclear palsy; vascular dementia; normal pressure hydrocephalus; traumatic spinal cord injury; HIV dementia; alcohol induced neurotoxicity; Down's syndrome; movement disorders of the central and/or peripheral nervous system; motor neurone diseases (MND); spinal muscular atrophy; or any other related neurological or psychiatric neurodegenerative disease; brain damage; brain injury; brain dysfunction; dysgraphia; dysarthria; apraxia; agnosia; amnesia; dizziness; vertigo; coma; stroke; spinal cord damage; spinal cord injury; spinal cord disorders; central neuropathy; peripheral neuropathy; cranial nerve disorder; trigeminal neuralgia; tumors of the nervous system; infections of the brain or spinal cord; encephalitis; meningitis; prion disease; complex regional pain syndrome; an autonomic nervous system disorder; autonomic neuropathy; dysautonomia; postural orthostatic tachycardia syndrome (POTS); neurocardiogenic syncope (NCS); multiple system atrophy (MSA); hereditary sensory and autonomic neuropathy (HSAN); Holmes-Adie syndrome (HAS); a sleep disorder; narcolepsy; pain; migraine; cluster headache; tension headache; back pain; lower back pain; neck pain; neuropathic pain; cancer pain; allodynia; arthritic pain; inflammatory pain; a neuropsychiatric disorder; attention deficit hyperactivity disorder; autism; Tourette's Syndrome; obsessive compulsive disorder; an autism spectrum disorder; Rett syndrome; Fragile X syndrome; Angelman syndrome; hyperkinetic disorder; mitochondrial disease; dystonia; a cancer; brain cancer; glioma; breast cancer; liver cancer; lung cancer; pancreatic cancer; melanoma; ovarian cancer; gastric cancer; renal cancer; bladder cancer; addiction; nicotine addiction; smoking; alcohol addiction; drug addiction; cannabis use disorder; a mental disorder; post-traumatic stress disorder; anxiety; early psychosis; schizophrenia; a cognitive disorder; stroke; cardiac ischemia; coronary artery disease; thromboembolism; myocardial infarction; ischemic related disease; a gastrointestinal disorder; inflammatory bowel disease; Crohn's disease; ulcerative colitis; nausea; vomiting; emesis; motion sickness; chemotherapy induced nausea; chemotherapy induced nausea vomiting; inflammation; arthritis; rheumatoid arthritis; osteoarthritis; diabetes; high blood pressure; poor insulin control; appetite suppression; anorexia; neonatal hypoxic-ischemic encephalopathy (NHIE); a degenerative skeletal muscle disease; or Duchenne muscular dystrophy (DMD).

In a further embodiment of the invention the epilepsy is Dravet syndrome, Lennox Gastaut syndrome, febrile infection related epilepsy syndrome (FIRES), Doose syndrome, Sturge Weber syndrome, CDKL5 mutation; Aicardi syndrome; bilateral polymicrogyria; Dup15q; SNAP25; benign rolandic epilepsy; juvenile myoclonic epilepsy; infantile spasm (West syndrome); and Landau-Kleffner syndrome, refractory epilepsy, juvenile spasms, West syndrome, infantile spasms, refractory infantile spasms, tuberous sclerosis complex (TSC); neurogenetic storage disorder, neuronal ceroid lipofuscinoses (NCL), Batten disease, brain abnormality, atonic, idiopathic, absence seizure, partial seizure, simple partial seizure, or complex partial seizure.

### BRIEF DESCRIPTION OF THE DRAWINGS

FIG. 1 depicts the biosynthetic pathway of cannabinoid production.

FIG. 2 depicts an autofluorescence spectra of botanically derived purified CBDV and synthetic CBDV at 100 mM representative of the excitation scan from 230 nm to below the emission wavelengths fixed at 400 or 440 nm.

FIG. 3 depicts an autofluorescence spectra of botanically derived purified CBDV and synthetic CBDV at 100 mM representative of the emission scan up to 800 nm above the excitation wavelengths fixed at 328/334/344 nm.

FIG. 4 depicts an autofluorescence spectra of botanically derived purified CBDV and synthetic CBDV at 100 mM representative of the excitation scan from 230 nm to below the emission wavelengths fixed at 400 or 440 nm.

FIG. 5 depicts an autofluorescence spectra of botanically derived purified CBDV and synthetic CBDV at 100 mM representative of the emission scan up to 800 nm above the excitation wavelengths fixed at 326/370 nm.

FIG. 6 depicts novel versus familiar exploration times in rats treated with botanically derived purified CBDV.

FIG. 7 depicts novel versus familiar exploration times in rats treated with synthetic CBDV.

### DEFINITIONS

In this application, unless otherwise clear from context, (i) the term “a” may be understood to mean “at least one”; (ii) the term “or” may be understood to mean “and/or”; (iii) the terms “comprising” and “including” may be understood to encompass itemized components or steps whether presented by themselves or together with one or more additional components or steps; and (iv) the terms “about” and “approximately” may be understood to permit standard variation as would be understood by those of ordinary skill in the art; and (v) where ranges are provided, endpoints are included.

About or Approximately: The terms “about” or “approximately”, as applied to one or more values of interest, refers to a value that is similar to a stated reference value. In certain embodiments, the term “about” or “approximately” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context.

Administration: As used herein, the term “administration” typically refers to the administration of a composition to a subject or system. Those of ordinary skill in the art will be aware of a variety of routes that may, in appropriate circumstances, be utilized for administration to a subject, for example a human. For example, in some embodiments, administration may be ocular, oral, parenteral, topical, etc. In some particular embodiments, administration may be bronchial (e.g., by bronchial instillation), buccal, dermal (which may be or comprise, for example, one or more of topical to the dermis, intradermal, interdermal, transdermal, etc.), enteral, intra-arterial, intradermal, intragastric, intramedullary, intramuscular, intranasal, intraperitoneal, intrathecal, intravenous, intraventricular, within a specific organ (e. g. intrahepatic), mucosal, nasal, oral, rectal, subcutaneous, sublingual, topical, tracheal (e.g., by intratracheal instillation), vaginal, vitreal, etc. In some embodiments, administration may involve dosing that is intermittent (e.g., a plurality of doses separated in time) and/or periodic (e.g., individual doses separated by a common period of time) dosing. In some embodiments, administration may involve continuous dosing (e.g., perfusion) for at least a selected period of time.

Agent: In general, the term “agent”, as used herein, may be used to refer to a compound or entity of any chemical class including, for example, a polypeptide, nucleic acid, saccharide, lipid, small molecule, metal, or combination or complex thereof. In appropriate circumstances, as will be clear from context to those skilled in the art, the term may be utilized to refer to an entity that is or comprises a cell or organism, or a fraction, extract, or component thereof. Alternatively, or additionally, as context will make clear, the term may be used to refer to a natural product in that it is found in and/or is obtained from nature. In some instances, again as will be clear from context, the term may be used to refer to one or more entities that is man-made in that it is designed, engineered, and/or produced through action of the hand of man and/or is not found in nature. In some embodiments, an agent may be utilized in isolated or pure form; in some embodiments, an agent may be utilized in crude form. In some embodiments, potential agents may be provided as collections or libraries, for example that may be screened to identify or characterize active agents within them. In some cases, the term “agent” may refer to a compound or entity that is or comprises a polymer; in some cases, the term may refer to a compound or entity that comprises one or more polymeric moieties. In some embodiments, the term “agent” may refer to a compound or entity that is not a polymer and/or is substantially free of any polymer and/or of one or more particular polymeric moieties. In some embodiments, the term may refer to a compound or entity that lacks or is substantially free of any polymeric moiety.

Amelioration: as used herein, refers to the prevention, reduction or palliation of a state, or improvement of the state of a subject. Amelioration includes but does not require complete recovery or complete prevention of a disease, disorder or condition (e.g., radiation injury).

Biologically active: as used herein, refers to an observable biological effect or result achieved by an agent or entity of interest. For example, in some embodiments, a specific binding interaction is a biological activity. In some embodiments, modulation (e.g., induction, enhancement, or inhibition) of a biological pathway or event is a biological activity. In some embodiments, presence or extent of a biological activity is assessed through detection of a direct or indirect product produced by a biological pathway or event of interest.

Cancer. The terms “cancer”, “malignancy”, “neoplasm”, “tumor”, and “carcinoma”, are used herein to refer to cells that exhibit relatively abnormal, uncontrolled, and/or autonomous growth, so that they exhibit an aberrant growth phenotype characterized by a significant loss of control of cell proliferation. In some embodiments, a tumor may be or comprise cells that are precancerous (e.g., benign), malignant, pre-metastatic, metastatic, and/or non-metastatic. The present disclosure specifically identifies certain cancers to which its teachings may be particularly relevant. In some embodiments, a relevant cancer may be characterized by a solid tumor. In some embodiments, a relevant cancer may be characterized by a hematologic tumor. In general, examples of different types of cancers known in the art include, for example, hematopoietic cancers including leukemias, lymphomas (Hodgkin's and non-Hodgkin's), myelomas and myeloproliferative disorders; sarcomas, melanomas, adenomas, carcinomas of solid tissue, squamous cell carcinomas of the mouth, throat, larynx, and lung, liver cancer, genitourinary cancers such as prostate, cervical, bladder, uterine, and endometrial cancer and renal cell carcinomas, bone cancer, pancreatic cancer, skin cancer, cutaneous or intraocular melanoma, cancer of the endocrine system, cancer of the thyroid gland, cancer of the parathyroid gland, head and neck cancers, breast cancer, gastro-intestinal cancers and nervous system cancers, benign lesions such as papilloma's, and the like.

Carrier as used herein, refers to a diluent, adjuvant, excipient, or vehicle with which a composition is administered. In some exemplary embodiments, carriers can include sterile liquids, such as, for example, water and oils, including oils of petroleum, animal, vegetable or synthetic origin, such as, for example, peanut oil, soybean oil, mineral oil, sesame oil and the like. In some embodiments, carriers are or include one or more solid components.

Comparable: As used herein, the term “comparable” refers to two or more agents, entities, situations, sets of conditions, etc., that may not be identical to one another but that are sufficiently similar to permit comparison there between so that one skilled in the art will appreciate that conclusions may reasonably be drawn based on differences or similarities observed. In some embodiments, comparable sets of conditions, circumstances, individuals, or populations are characterized by a plurality of substantially identical features and one or a small number of varied features. Those of ordinary skill in the art will understand, in context, what degree of identity is required in any given circumstance for two or more such agents, entities, situations, sets of conditions, etc. to be considered comparable. For example, those of ordinary skill in the art will appreciate that sets of circumstances, individuals, or populations are comparable to one another when characterized by a sufficient number and type of substantially identical features to warrant a reasonable conclusion that differences in results obtained or phenomena observed under or with different sets of circumstances, individuals, or populations are caused by or indicative of the variation in those features that are varied.

Composition: Those skilled in the art will appreciate that the term “composition” may be used to refer to a discrete physical entity that comprises one or more specified components. In general, unless otherwise specified, a composition may be of any form—e.g., gas, gel, liquid, solid, etc.

Comprising: A composition or method described herein as “comprising” one or more named elements or steps is open-ended, meaning that the named elements or steps are essential, but other elements or steps may be added within the scope of the composition or method. To avoid prolixity, it is also understood that any composition or method described as “comprising” (or which “comprises”) one or more named elements or steps also describes the corresponding, more limited composition or method “consisting essentially of” (or which “consists essentially of”) the same named elements or steps, meaning that the composition or method includes the named essential elements or steps and may also include additional elements or steps that do not materially affect the basic and novel characteristic(s) of the composition or method. It is also understood that any composition or method described herein as “comprising” or “consisting essentially of” one or more named elements or steps also describes the corresponding, more limited, and closed-ended composition or method “consisting of” (or “consists of”) the named elements or steps to the exclusion of any other unnamed element or step. In any composition or method disclosed herein, known or disclosed equivalents of any named essential element or step may be substituted for that element or step.

Determine: Many methodologies described herein include a step of “determining”. Those of ordinary skill in the art, reading the present specification, will appreciate that such “determining” can utilize or be accomplished through use of any of a variety of techniques available to those skilled in the art, including for example specific techniques explicitly referred to herein. In some embodiments, determining involves manipulation of a physical sample. In some embodiments, determining involves consideration and/or manipulation of data or information, for example utilizing a computer or other processing unit adapted to perform a relevant analysis. In some embodiments, determining involves receiving relevant information and/or materials from a source. In some embodiments, determining involves comparing one or more features of a sample or entity to a comparable reference.

Dosage form or unit dosage form: Those skilled in the art will appreciate that the term “dosage form” may be used to refer to a physically discrete unit of an active agent (e.g., a therapeutic or diagnostic agent) for administration to a subject. Typically, each such unit contains a predetermined quantity of active agent. In some embodiments, such quantity is a unit dosage amount (or a whole fraction thereof) appropriate for administration in accordance with a dosing regimen that has been determined to correlate with a desired or beneficial outcome when administered to a relevant population (i.e., with a therapeutic dosing regimen). Those of ordinary skill in the art appreciate that the total amount of a therapeutic composition or agent administered to a particular subject is determined by one or more attending physicians and may involve administration of multiple dosage forms.

Dosing regimen: Those skilled in the art will appreciate that the term “dosing regimen” may be used to refer to a set of unit doses (typically more than one) that are administered individually to a subject, typically separated by periods of time. In some embodiments, a given therapeutic agent has a recommended dosing regimen, which may involve one or more doses. In some embodiments, a dosing regimen comprises a plurality of doses each of which is separated in time from other doses. In some embodiments, individual doses are separated from one another by a time period of the same length; in some embodiments, a dosing regimen comprises a plurality of doses and at least two different time periods separating individual doses. In some embodiments, all doses within a dosing regimen are of the same unit dose amount. In some embodiments, different doses within a dosing regimen are of different amounts. In some embodiments, a dosing regimen comprises a first dose in a first dose amount, followed by one or more additional doses in a second dose amount different from the first dose amount. In some embodiments, a dosing regimen comprises a first dose in a first dose amount, followed by one or more additional doses in a second dose amount same as the first dose amount. In some embodiments, a dosing regimen is correlated with a desired or beneficial outcome when administered across a relevant population (i.e., is a therapeutic dosing regimen).

Excipient: as used herein, refers to a non-therapeutic agent that may be included in a pharmaceutical composition, for example to provide or contribute to a desired consistency or stabilizing effect. Suitable pharmaceutical excipients include, for example, starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like.

Improve, increase, inhibit or reduce: As used herein, these terms, or grammatically comparable comparative terms, indicate values that are relative to a comparable reference measurement. For example, in some embodiments, an assessed value achieved with an agent of interest may be “improved” relative to that obtained with a comparable reference agent. Alternatively or additionally, in some embodiments, an assessed value achieved in a subject or system of interest may be “improved” relative to that obtained in the same subject or system under different conditions (e.g., prior to or after an event such as administration of an agent of interest), or in a different, comparable subject (e.g., in a comparable subject or system that differs from the subject or system of interest in presence of one or more indicators of a particular disease, disorder or condition of interest, or in prior exposure to a condition or agent, etc.). In some embodiments, comparative terms refer to statistically relevant differences (e.g., that are of a prevalence and/or magnitude sufficient to achieve statistical relevance). Those skilled in the art will be aware, or will readily be able to determine, in a given context, a degree and/or prevalence of difference that is required or sufficient to achieve such statistical significance.

In vitro: The term “in vitro” as used herein refers to events that occur in an artificial environment, e.g., in a test tube or reaction vessel, in cell culture, etc., rather than within a multi-cellular organism.

In vivo: as used herein refers to events that occur within a multi-cellular organism, such as a human and a non-human animal. In the context of cell-based systems, the term may be used to refer to events that occur within a living cell (as opposed to, for example, in vitro systems).

Isolated: as used herein, refers to a substance and/or entity that has been (1) separated from at least some of the components with which it was associated when initially produced (whether in nature and/or in an experimental setting), and/or (2) designed, produced, prepared, and/or manufactured by the hand of man. In some embodiments, isolated agents are about 80%, about 85%, about 90%, about 91%, about 92%, about 93%, about 94%, about 95%, about 96%, about 97%, about 98%, about 99%, or more than about 99% pure. As used herein, a substance is “pure” if it is substantially free of other components.

Isomer: As is known in the art, many chemical entities (in particular many organic molecules and/or many small molecules) can exist in a variety of structural (e.g., geometric/conformational) and/or optical isomeric forms. For example, any chiral center can exist in R and S configurations; double bonds can exist in Z and E conformational isomers, certain structural elements can adopt two or more tautomeric forms, etc. In some embodiments, as will be clear to those skilled in the art from context, depiction of or reference to a particular compound structure herein may represent all structural and/or optical isomers thereof. In some embodiments, as will be clear to those skilled in the art from context, depiction of or reference to a particular compound structure herein is intended to encompass only the depicted or referenced isomeric form. In some embodiments, compositions including a chemical entity that can exist in a variety of isomeric forms include a plurality of such forms; in some embodiments such compositions include only a single form. For example, in some embodiments, compositions including a chemical entity that can exist as a variety of optical isomers (e.g., stereoisomers, diastereomers, etc.) include a racemic population of such optical isomers; in some embodiments such compositions include only a single optical isomer and/or include a plurality of optical isomers that together retain optical activity. Where there exists two or more isomers within a composition they may exist as a mixture with various ratios.

Mixture: The phrase “mixture” describes a combination of two or more different compounds or agents which occur within the same composition.

Moiety: Those skilled in the art will appreciate that a “moiety” is a defined chemical group or entity with a particular structure and/or or activity, as described herein.

Oral: The phrases “oral administration” and “administered orally” as used herein have their art-understood meaning referring to administration by mouth of a compound or composition.

Parenteral: The phrases “parenteral administration” and “administered parenterally” as used herein have their art-understood meaning referring to modes of administration other than enteral and topical administration, usually by injection, and include, without limitation, intravenous, intramuscular, intraarterial, intrathecal, intracapsular, intraorbital, intracardiac, intradermal, intraperitoneal, transtracheal, subcutaneous, subcuticular, intraarticulare, subcapsular, subarachnoid, intraspinal, and intrasternal injection and infusion.

Patient: As used herein, the term “patient” refers to any organism to which a provided composition is or may be administered, e.g., for experimental, diagnostic, prophylactic, cosmetic, and/or therapeutic purposes. Typical patients include animals (e.g., mammals such as mice, rats, rabbits, non-human primates, and/or humans). In some embodiments, a patient is a human. In some embodiments, a patient is suffering from or susceptible to one or more disorders or conditions. In some embodiments, a patient displays one or more symptoms of a disorder or condition. In some embodiments, a patient has been diagnosed with one or more disorders or conditions. In some embodiments, the disorder or condition is or includes cancer, or presence of one or more tumors. In some embodiments, the patient is receiving or has received certain therapy to diagnose and/or to treat a disease, disorder, or condition.

Pharmaceutical composition: As used herein, the term “pharmaceutical composition” refers to an active agent, formulated together with one or more pharmaceutically acceptable carriers. In some embodiments, active agent is present in unit dose amount appropriate for administration in a therapeutic regimen that shows a statistically significant probability of achieving a predetermined therapeutic effect when administered to a relevant population. In some embodiments, pharmaceutical compositions may be specially formulated for administration in solid or liquid form, including those adapted for the following: oral administration, for example, drenches (aqueous or non-aqueous solutions or suspensions), tablets, e.g., those targeted for buccal, sublingual, and systemic absorption, boluses, powders, granules, pastes for application to the tongue; parenteral administration, for example, by subcutaneous, intramuscular, intravenous or epidural injection as, for example, a sterile solution or suspension, or sustained-release formulation; topical application, for example, as a cream, ointment, or a controlled-release patch or spray applied to the skin, lungs, or oral cavity; intravaginally or intrarectally, for example, as a pessary, cream, or foam; sublingually; ocularly; transdermally; or nasally, pulmonary, and to other mucosal surfaces.

Pharmaceutically acceptable: As used herein, the phrase “pharmaceutically acceptable” refers to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

Pharmaceutically acceptable carrier: As used herein, the term “pharmaceutically acceptable carrier” means a pharmaceutically-acceptable material, composition or vehicle, such as a liquid or solid filler, diluent, excipient, or solvent encapsulating material, involved in carrying or transporting the subject compound from one organ, or portion of the body, to another organ, or portion of the body. Each carrier must be “acceptable” in the sense of being compatible with the other ingredients of the formulation and not injurious to the patient. Some examples of materials which can serve as pharmaceutically-acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; pH buffered solutions; polyesters, polycarbonates and/or polyanhydrides; and other non-toxic compatible substances employed in pharmaceutical formulations.

Predetermined: By predetermined is meant deliberately selected, for example as opposed to randomly occurring or achieved.

Prevent or prevention: as used herein when used in connection with the occurrence of a disease, disorder, and/or condition, refers to reducing the risk of developing the disease, disorder and/or condition and/or to delaying onset of one or more characteristics or symptoms of the disease, disorder or condition. Prevention may be considered complete when onset of a disease, disorder or condition has been delayed for a predefined period of time.

Predominantly present: The term “predominantly present”, as used herein, refers to the quantity of an entity (e.g., a specific cannabinoid or isomer thereof) in a preparation or composition. For example, a cannabinoid may be predominantly present if it is at least about 50%, about 55%, about 60%, about 65%, about 70%, about 75%, about 80%, about 85%, about 90%, about 95%, about 96%, about 97%, about 98%, about 99%, or about 100% of the total cannabinoid in the preparation or composition.

Prevention: The term “prevention”, as used herein, refers to a delay of onset, and/or reduction in frequency and/or severity of one or more symptoms of a particular disease, disorder or condition. In some embodiments, prevention is assessed on a population basis such that an agent is considered to “prevent” a particular disease, disorder or condition if a statistically significant decrease in the development, frequency, and/or intensity of one or more symptoms of the disease, disorder or condition is observed in a population susceptible to the disease, disorder, or condition. Prevention may be considered complete when onset of a disease, disorder or condition has been delayed for a predefined period of time.

Pure: As used herein, an agent or entity is “pure” if it is substantially free of other components. For example, a preparation that contains more than about 90% of a particular agent or entity is typically considered to be a pure preparation. In some embodiments, an agent or entity is at least 91%, at least 92%, at least 93%, at least 94%, at least 95%, at least 96%, at least 97%, at least 98%, or at least 99% pure.

Reference: As used herein describes a standard or control relative to which a comparison is performed. For example, in some embodiments, an agent, animal, individual, population, sample, sequence or value of interest is compared with a reference or control agent, animal, individual, population, sample, sequence or value. In some embodiments, a reference composition may comprise one or more synthetic cannabinoids. In some embodiments a reference composition may contain different types of cannabinoids, different isomeric forms of cannabinoids, different distribution of cannabinoids, different quantities of cannabinoids, etc. as compared to a test composition. In some embodiments, a reference or control is tested and/or determined substantially simultaneously with the testing or determination of interest. In some embodiments, a reference or control is a historical reference or control, optionally embodied in a tangible medium. Typically, as would be understood by those skilled in the art, a reference or control is determined or characterized under comparable conditions or circumstances to those under assessment. Those skilled in the art will appreciate when sufficient similarities are present to justify reliance on and/or comparison to a particular possible reference or control.

Response: As used herein, a response to treatment may refer to any beneficial alteration in a subject's condition that occurs as a result of or correlates with treatment. Such alteration may include stabilization of the condition (e.g., prevention of deterioration that would have taken place in the absence of the treatment), amelioration of symptoms of the condition, and/or improvement in the prospects for cure of the condition, etc. Response may be measured according to a wide variety of criteria, including clinical criteria and objective criteria. Techniques for assessing response include, but are not limited to, clinical examination, positron emission tomography, chest X-ray CT scan, MRI, ultrasound, endoscopy, laparoscopy, presence or level of tumor markers in a sample obtained from a subject, cytology, and/or histology. The exact response criteria can be selected in any appropriate manner, provided that when comparing groups of cells or subjects, the groups to be compared are assessed based on the same or comparable criteria for determining response rate. One of ordinary skill in the art will be able to select appropriate criteria.

Solid form: As is known in the art, many chemical entities (in particular many organic molecules and/or many small molecules) can adopt a variety of different solid forms such as, for example, amorphous forms and/or crystalline forms (e.g., polymorphs, hydrates, solvates, etc.). In some embodiments, such entities may be utilized as a single such form (e.g., as a pure preparation of a single polymorph). In some embodiments, such entities may be utilized as a mixture of such forms.

Subject: As used herein, the term “subject” refers an organism, typically a mammal (e.g., a human, in some embodiments including prenatal human forms). In some embodiments, a subject refers to any organism (e.g., mammals such as mice, rats, rabbits, non-human primates, and humans; insects; worms; etc.) and plants to which a provided compound or composition is administered in accordance with the present disclosure e.g., for experimental, diagnostic, prophylactic, and/or therapeutic purposes. In some embodiments, a subject is suffering from a relevant disease, disorder or condition. In some embodiments, a subject is susceptible to a disease, disorder, or condition. In some embodiments, a subject displays one or more symptoms or characteristics of a disease, disorder or condition. In some embodiments, a subject does not display any symptom or characteristic of a disease, disorder, or condition. In some embodiments, a subject is someone with one or more features characteristic of susceptibility to or risk of a disease, disorder, or condition. In some embodiments, a subject is a patient. In some embodiments, a subject is an individual to whom diagnosis and/or therapy is and/or has been administered.

Substantially: As used herein, the term “substantially” refers to the qualitative condition of exhibiting total or near-total extent or degree of a characteristic or property of interest. One of ordinary skill in the biological arts will understand that biological and chemical phenomena rarely, if ever, go to completion and/or proceed to completeness or achieve or avoid an absolute result. The term “substantially” is therefore used herein to capture the potential lack of completeness inherent in many biological and chemical phenomena.

Suffering from: An individual who is “suffering from” a disease, disorder, and/or condition has been diagnosed with and/or displays one or more symptoms of a disease, disorder, and/or condition.

Susceptible to: An individual who is “susceptible to” a disease, disorder, and/or condition is one who has a higher risk of developing the disease, disorder, and/or condition than does a member of the general public. In some embodiments, an individual who is susceptible to a disease, disorder and/or condition may not have been diagnosed with the disease, disorder, and/or condition. In some embodiments, an individual who is susceptible to a disease, disorder, and/or condition may exhibit symptoms of the disease, disorder, and/or condition. In some embodiments, an individual who is susceptible to a disease, disorder, and/or condition may not exhibit symptoms of the disease, disorder, and/or condition. In some embodiments, an individual who is susceptible to a disease, disorder, and/or condition will develop the disease, disorder, and/or condition. In some embodiments, an individual who is susceptible to a disease, disorder, and/or condition will not develop the disease, disorder, and/or condition.

Symptoms are reduced: According to the present invention, “symptoms are reduced” when one or more symptoms of a particular disease, disorder or condition is reduced in magnitude (e.g., intensity, severity, etc.) and/or frequency. For purposes of clarity, a delay in the onset of a particular symptom is considered one form of reducing the frequency of that symptom.

Systemic: The phrases “systemic administration,” “administered systemically,” “peripheral administration,” and “administered peripherally” as used herein have their art-understood meaning referring to administration of a compound or composition such that it enters the recipient's system.

Therapeutic agent: As used herein, the phrase “therapeutic agent” in general refers to any agent that elicits a desired pharmacological effect when administered to an organism. In some embodiments, an agent is considered to be a therapeutic agent if it demonstrates a statistically significant effect across an appropriate population. In some embodiments, the appropriate population may be a population of model organisms. In some embodiments, an appropriate population may be defined by various criteria, such as a certain age group, gender, genetic background, preexisting clinical conditions, etc. In some embodiments, a therapeutic agent is a substance that can be used to alleviate, ameliorate, relieve, inhibit, prevent, delay onset of, reduce severity of, and/or reduce incidence of one or more symptoms or features of a disease, disorder, and/or condition. In some embodiments, a “therapeutic agent” is an agent that has been or is required to be approved by a government agency before it can be marketed for administration to humans. In some embodiments, a “therapeutic agent” is an agent for which a medical prescription is required for administration to humans.

Therapeutic regimen: A “therapeutic regimen”, as that term is used herein, refers to a dosing regimen whose administration across a relevant population may be correlated with a desired or beneficial therapeutic outcome.

Therapeutically effective amount: As used herein, the term “therapeutically effective amount” means an amount of a substance (e.g., a therapeutic agent, active ingredient, preparation, composition, and/or formulation) that elicits a desired a desired effect (e.g., a desired biological, clinical, or pharmacological effect or response) when administered as part of a therapeutic regimen. In some embodiments, a therapeutically effective amount of a substance is an amount that is sufficient, when administered to a subject suffering from or susceptible to a disease, disorder, and/or condition, to treat, diagnose, prevent, reduce the severity of, stabilize one or more characteristics of, and/or delay the onset of the disease, disorder, and/or condition. In some embodiments, the term refers to an amount sufficient to produce the effect in at least a significant percentage (e.g., at least about 25%, about 30%, about 40%, about 50%, about 60%, about 70%, about 80%, about 90%, about 95%, or more) of a population that is suffering from and/or susceptible to a disease, disorder, and/or condition. As will be appreciated by those of ordinary skill in this art, the effective amount of a substance may vary depending on such factors as the desired biological endpoint, the substance to be delivered, the target cell or tissue, etc. For example, the effective amount of compound in a formulation to treat a disease, disorder, and/or condition is the amount that alleviates, ameliorates, relieves, inhibits, prevents, delays onset of, reduces severity of and/or reduces incidence of one or more symptoms or features of the disease, disorder, and/or condition. In some embodiments, a therapeutically effective amount is administered in a single dose; in some embodiments, multiple unit doses are required to deliver a therapeutically effective amount.

Treat: As used herein, the term “treat,” “treatment,” or “treating” refers to any method used to partially or completely alleviate, ameliorate, relieve, inhibit, prevent, delay onset of, reduce severity of, and/or reduce incidence of one or more symptoms or features of a disease, disorder, and/or condition. In some embodiments, treatment refers to administration of a therapy that partially or completely alleviates, ameliorates, relives, inhibits, delays onset of, reduces severity of, and/or reduces incidence of one or more symptoms, features, and/or causes of a particular disease, disorder, and/or condition. Treatment may be administered to a subject who does not exhibit signs of a disease, disorder, and/or condition. In some embodiments, treatment may be administered to a subject who exhibits only early signs of the disease, disorder, and/or condition, for example for the purpose of decreasing the risk of developing pathology associated with the disease, disorder, and/or condition.

Unit dose: The expression “unit dose” as used herein refers to an amount administered as a single dose and/or in a physically discrete unit of a pharmaceutical composition. In many embodiments, a unit dose contains a predetermined quantity of an active agent. In some embodiments, a unit dose contains an entire single dose of the agent. In some embodiments, more than one unit dose is administered to achieve a total single dose. In some embodiments, administration of multiple unit doses is required, or expected to be required, in order to achieve an intended effect. A unit dose may be, for example, a volume of liquid (e.g., an acceptable carrier) containing a predetermined quantity of one or more therapeutic agents, a predetermined amount of one or more therapeutic agents in solid form, a sustained release formulation or drug delivery device containing a predetermined amount of one or more therapeutic agents, etc. It will be appreciated that a unit dose may be present in a formulation that includes any of a variety of components in addition to the therapeutic agent(s). For example, acceptable carriers (e.g., pharmaceutically acceptable carriers), diluents, stabilizers, buffers, preservatives, etc., may be included as described infra. It will be appreciated by those skilled in the art, in many embodiments, a total appropriate daily dosage of a particular therapeutic agent may comprise a portion, or a plurality, of unit doses, and may be decided, for example, by the attending physician within the scope of sound medical judgment. In some embodiments, the specific effective dose level for any particular subject or organism may depend upon a variety of factors including the disorder being treated and the severity of the disorder; activity of specific active compound employed; specific composition employed; age, body weight, general health, sex and diet of the subject; time of administration, and rate of excretion of the specific active compound employed; duration of the treatment; drugs and/or additional therapies used in combination or coincidental with specific compound(s) employed, and like factors well known in the medical arts.

### DETAILED DESCRIPTION

Cannabinoids are natural and synthetic compounds structurally or pharmacologically related to the constituents of the cannabis plant or to the endogenous agonists (endocannabinoids) of the cannabinoid receptors CB1 or CB2. The only way in nature in which these compounds are produced is by the cannabis plant. Cannabis is a genus of flowering plants in the family Cannabaceae, comprising the species Cannabis sativa, Cannabis indica, and Cannabis ruderalis (sometimes considered as part of Cannabis sativa).

Cannabis plants comprise a highly complex mixture of compounds. At least 568 unique molecules have been identified. Among these compounds are cannabinoids, terpenoids, sugars, fatty acids, flavonoids, other hydrocarbons, nitrogenous compounds, and amino acids.

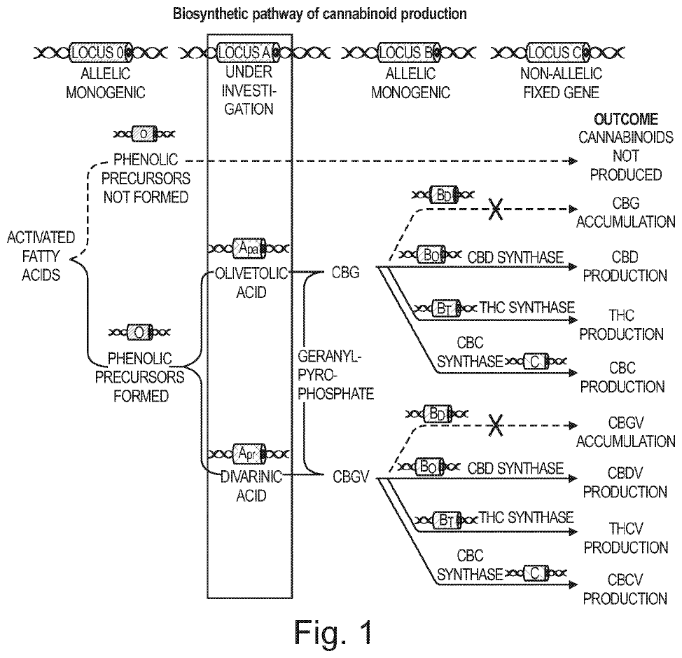

Cannabinoids exert their physiological effects through a variety of receptors including, but not limited to, adrenergic receptors, cannabinoid receptors (CB1 and CB2), GPR55, GPR3, or GPR5. The principle cannabinoids present in cannabis plants are cannabinoid acids tetrahydrocannabinolic acid (THCA) and cannabidiolic acid (CBDA) with small amounts of their respective neutral (decarboxylated) cannabinoids. In addition, cannabis may contain lower levels of other minor cannabinoids. “Chemical composition, pharmacological profiling, and complete physiological effects of these medicinal plants, and more importantly the extracts from cannabis, remain to be fully understood.” Lewis, M. M. et al., ACS Omega, 2, 6091-6103 (2017). FIG. 1 depicts an exemplary schematic of the biosynthetic pathways of certain phytocannabinoids.

Crude extracts from cannabis plants containing CBDV have been used by patients suffering from diseases and disorders. However, such crude products are unsuitable for use in pharmaceutical formulations. Those seeking to prepare more consistent CBDV preparations for use in treating diseases or disorders have made a concerted effort to either prepare CBDV synthetically or attempt to remove all compounds other than CBDV, particularly psychoactive compounds such as THC, from plant derived cannabinoids.

The present invention encompasses the surprising discovery that a botanically derived purified CBDV preparation, comprising one or more additional cannabinoids, and suitable for pharmaceutical use, exhibits enhanced therapeutic efficacy when compared to prior CBD preparations which differ from the composition disclosed herein.

These preparations differ either by being purified to the extent that no other impurities exist or being produced synthetically thereby comprising no additional cannabinoids that would be produced by nature or further differ by being an unpurified plant extract which extract comprises some or all of the cannabinoids and non-cannabinoid compounds that are co-produced by the plant and co-extracted in the preparation of the extract. In some embodiments botanically derived purified CBDV preparation of the present invention may be administered in a lower dose of CBDV than a synthetic or completely pure preparation of CBDV.

### Cannabinoids

As stated, cannabinoids are a class of compounds which may be derived naturally from the cannabis plant or produced synthetically via chemical synthesis.

More than 100 different cannabinoids produced by cannabis have been identified. These cannabinoids can be split into different groups as follows: phytocannabinoids; endocannabinoids and synthetic cannabinoids (which may be novel cannabinoids or synthetically produced versions of phytocannabinoids or endocannabinoids).

Phytocannabinoids are cannabinoids that originate from nature and can be found in the cannabis plant. Phytocannabinoids can be isolated from plants to produce a highly purified extract. Phytocannabinoids may be obtained as either the neutral (decarboxylated form) or the carboxylic acid form depending on the method used to extract the cannabinoids from plant material. For example, it is known that heating the carboxylic acid form will cause most of the carboxylic acid form to decarboxylate into the neutral form. Phytocannabinoids can only be produced from plants, however versions of phytocannabinoids may be produced synthetically via chemical synthesis.

Endocannabinoids are endogenous lipid-based retrograde neurotransmitters that bind to cannabinoid receptors, and cannabinoid receptor proteins that are expressed throughout the mammalian central nervous system (including the brain) and peripheral nervous system. The endocannabinoid system is involved in regulating a variety of physiological and cognitive processes including fertility, pregnancy, during pre- and postnatal development, appetite, pain-sensation, mood, and memory, and in mediating the pharmacological effects of cannabis.

“Synthetic cannabinoids” are compounds that have a cannabinoid-like structure and are manufactured using chemical means rather than by the plant.

Certain cannabinoids are described in more detail below. Although little is known about these cannabinoids, CBDV preparations and compositions described herein which comprise one or more of these components show surprising efficacy, particularly when compared with pure and/or synthetic CBDV compositions.

### Cannabidiol (CBD)

CBD is a major cannabinoid constituent of Cannabis species, such as the hemp plant (Cannabis sativa). Unlike other cannabinoids, such as THC, CBD does not bind CB1 or CB2, or its binding to the receptors is negligible in terms of inducing a pharmacological effect. Thus, CBD does not cause the central or peripheral nervous system effects mediated by the CB1 or CB2 receptors. CBD has little or no psychotropic (cannabimimetic) activity and its molecular structure and properties are substantially different from those of other cannabinoids.

CBD administration has been the subject of research in an attempt to provide an alternative treatment for various diseases and disorders which may respond to such treatment.

In some embodiments CBD is isolated from a cannabis plant. In some embodiments CBD is prepared synthetically. In some embodiments, CBD is present as (−)-trans-CBD.

### Tetrahydrocannabinol (THC)

THC is the principal psychoactive constituent of cannabis.

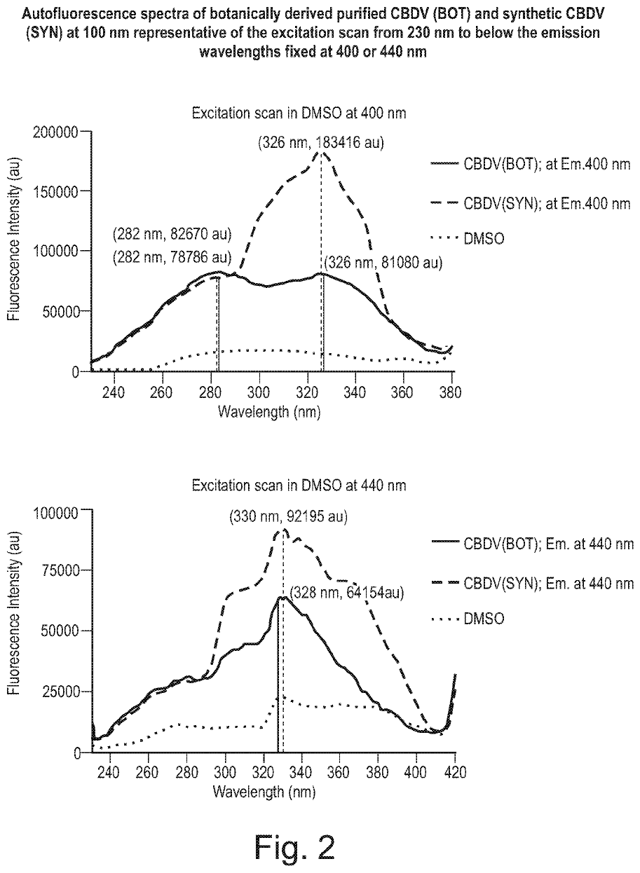

The THC molecule may exist as four distinct chiral forms as shown in FIG. 2. THC has 2 stereocenters which in turn enable the existence of 4 stereoisomers:(+)-trans-THC; (−)-trans-THC; (+)-cis-THC and (−)-cis-THC. THC commonly occurs in nature as the (−)-trans-THC isomer (Hollister, 1970)

The THC molecule mostly occurs as (−)-trans-Δ⁹-tetrahydrocannabinol, however the (−)-trans-Δ⁸-tetrahydrocannabinol homolog is also known to exist. The skilled person will appreciate that reference to the compound THC may refer to either the Δ⁸ or the Δ⁹ homolog.

In some embodiments THC is isolated from a cannabis plant. In some embodiments THC is prepared synthetically. In some embodiments, THC is present as (−)-trans-THC. In some embodiments, THC is present as (−)-cis-THC. In some embodiments, THC is present as (+)-trans-THC. In some embodiments, THC is present as (+)-cis-THC.

In some embodiments the THC is present as a mixture of isomers. In some embodiments the mixture will comprise one or more of (+)-trans-THC, (−)-trans-THC, (+)-cis-THC and (−)-cis-THC.

### Cannabidivarin (CBDV)

CBDV is a homolog of CBD, with the side-chain shortened by two methylene bridges. In some embodiments CBDV is isolated from a cannabis plant. In some embodiments CBDV is prepared synthetically. In some embodiments, CBDV is present as (−)-trans-CBDV.

Cannabidivarin acid (CBDVA)

CBDVA is the undecarboxylated form of CBDV. In some embodiments CBDVA is isolated from a cannabis plant. In some embodiments CBDVA is prepared synthetically. In some embodiments, CBDVA is present as (−)-trans-CBDVA.

### Tetrahydrocannabivarin (THCV)

THCV is a homolog of THC, with the side-chain shortened by two methylene bridges. In some embodiments THCV is isolated from a cannabis plant. In some embodiments THCV is prepared synthetically. In some embodiments, THCV is present as (−)-trans-THCV.

### Cannabidiol-C1 (CBD-C1)

In some embodiments CBD-C1 is isolated from a cannabis plant. In some embodiments CBD-C1 is prepared synthetically. In some embodiments, CBD-C1 is present as (−)-trans-CBD-C1.

### Cannabidiol-C4 (CBD-C4)

In some embodiments CBD-C4 is isolated from a cannabis plant. In some embodiments CBD-C4 is prepared synthetically. In some embodiments, CBD-C4 is present as (−)-trans-CBD-C4.

### CBDV Preparations

The present disclosure provides certain CBDV preparations, characterized by chemical components and/or functional properties that distinguish them from prior CBDV compositions.

In some embodiments, a CBDV preparation comprises about 90%, about 91%, about 92%, about 93%, about 94%, about 95%, about 96%, about 97%, about 98%, about 99%, or more than about 99% of the other components with which they were initially associated. Preferably the CBDV preparation comprises at least 95% CBDV based on total amount of cannabinoid in the preparation.

In some embodiments, the CBDV comprises (−)-trans-CBD isoform.

In some embodiments, the CBDV preparation further comprises tetrahydrocannabinol (THC). In some embodiments, a CBDV preparation comprises up to about 1%, about 2%, about 3%, about 4%, or about 5% THC based on total amount of cannabinoid in the preparation. In some embodiments, a CBDV preparation comprises not more than 0.1% THC based on total amount of cannabinoid in the preparation. In some embodiments, a CBDV preparation comprises about 0.01% to about 0.1% THC based on total amount of cannabinoid in the preparation. In some embodiments, a CBDV preparation comprises about 0.02% to about 0.05% THC based on total amount of cannabinoid in the preparation. In some embodiments, a CBDV preparation comprises at least about 0.1% THC based on total amount of cannabinoid in the preparation. In some embodiments, a CBDV preparation comprises at least about 0.02% THC based on total amount of cannabinoid in the preparation. In some embodiments, the THC comprises Δ⁹-THC.

In some embodiments, the THC is present as a mixture of different isomers. In some embodiments, the THC comprises trans-THC and cis-THC. In some embodiments, the trans-THC and cis-THC are present at a ratio of about 5:1 (trans-THC:cis-THC). In some embodiments, the trans-THC and cis-THC are present at a ratio of about 3.5:1 (trans-THC:cis-THC). In some embodiments, the trans-THC and cis-THC are present at a ratio of about 2:1 (trans-THC:cis-THC). In some embodiments, the trans-THC and cis-THC are present at a ratio of about 1:1 (trans-THC:cis-THC). In some embodiments, the trans-THC and cis-THC are present at a ratio of about 0.8:1 (trans-THC:cis-THC).

In some embodiments the cis-THC is present as a mixture of (−)-cis-THC and (+)-cis-THC. In some embodiments, the (−)-cis-THC and (+)-cis-THC are present at a ratio of about 20:1 to 1:20 ((−)-cis-THC:(+)-cis-THC). In some embodiments, the (−)-cis-THC and (+)-cis-THC are present at a ratio of about 15:1 to 1:15 ((−)-cis-THC:(+)-cis-THC). In some embodiments, the (−)-cis-THC and (+)-cis-THC are present ata ratio of about 10:1 to 1:10 ((−)-cis-THC:(+)-cis-THC). In some embodiments, the (−)-cis-THC and (+)-cis-THC are present at a ratio of about 9:1 to 1:9 ((−)-cis-THC:(+)-cis-THC). In some embodiments, the (−)-cis-THC and (+)-cis-THC are present at a ratio of about 5:1 to 1:5 ((−)-cis-THC:(+)-cis-THC). In some embodiments, the (−)-cis-THC and (+)-cis-THC are present ata ratio of about 3:1 to 1:3 ((−)-cis-THC:(+)-cis-THC). In some embodiments, the (−)-cis-THC and (+)-cis-THC are present at a ratio of about 2:1 to 1:2 ((−)-cis-THC:(+)-cis-THC). In some embodiments, the (−)-cis-THC and (+)-cis-THC are present at a ratio of about 1:1 ((−)-cis-THC:(+)-cis-THC). In some embodiments, the (−)-cis-THC and (+)-cis-THC are present at a ratio of about 9:1 ((−)-cis-THC:(+)-cis-THC).

In some embodiments, a CBDV preparation comprises one or more cannabinoids other than THC. In some embodiments, a CBDV preparation comprises no more than 5% cannabinoids other than CBDV based on total amount of cannabinoid in the preparation.

In some embodiments, a CBDV preparation comprises cannabidiol (CBD). In some embodiments, the CBD comprises the (−)-trans-CBD isoform. In some embodiments, a CBDV preparation comprises about 1% to about 5% CBD based on total amount of cannabinoid in the preparation.

In some embodiments, a CBDV preparation comprises CBD-C4 (CBD-C4). In some embodiments, the CBD-C4 comprises (−)-trans-CBD-C4 isoform. In some embodiments, a CBDV preparation comprises about 0.1% to about 0.4% CBD-C4 based on total amount of cannabinoid in the preparation.

In some embodiments, a CBDV preparation comprises CBD-C1 (CBD-C1). In some embodiments, the CBD-C1 comprises (−)-trans-CBD-C1 isoform. In some embodiments, a CBD preparation comprises about 0.1% to about 0.25% CBD-C1 based on total amount of cannabinoid in the preparation.

In some embodiments, a CBDV preparation comprises cannabidivarin acid (CBDVA). In some embodiments, the CBDVA comprises (−)-trans-CBDVA isoform. In some embodiments, a CBDV preparation comprises about 0.1% to about 0.25% CBDVA based on total amount of cannabinoid in the preparation.

In some embodiments, a CBDV preparation comprises tetrahydrocannabivarin (THCV). In some embodiments, the THCV comprises (−)-trans-THCV isoform. In some embodiments, a CBDV preparation comprises about 0.001% to about 0.008% CBDVA based on total amount of cannabinoid in the preparation.

In some embodiments, at least a portion of at least one of the cannabinoids present in a CBDV preparation is isolated from cannabis plant material. In some embodiments, at least a portion of the CBDV present in a CBDV preparation is isolated from cannabis plant material. In some embodiments, at least a portion of the THC present in a CBDV preparation is isolated from cannabis plant material. In some embodiments, substantially all of at least one of the cannabinoids present in a CBDV preparation is isolated from cannabis plant material. In some embodiments, substantially all the CBDV present in a CBDV preparation is isolated from cannabis plant material. In some embodiments, substantially all the THC present in a CBDV preparation is isolated from cannabis plant material. In some embodiments, substantially all of the cannabinoids present in a CBDV preparation are isolated from cannabis plant material. In some embodiments, the cannabis plant material is from a Cannabis sativa, Cannabis indica, or Cannabis ruderalis plant. In some embodiments, the cannabis plant is a high-CBDV containing cannabis chemotype. In some embodiments, the cannabis plant is a high-CBDV containing cannabis chemotype of Cannabis sativa L. In some embodiments, the cannabis plant material comprises about 5% to about 20% CBDV based on total amount of cannabinoid in the preparation. In some embodiments, the cannabis plant material comprises about 10% to about 15% CBDV based on total amount of cannabinoid in the preparation. In some embodiments, the cannabis plant material comprises trans-THC and cis-THC are present at a ratio of about 3.5:1 (trans-THC:cis-THC). In some embodiments, the cannabis plant material comprises trans-THC and cis-THC are present at a ratio of about 0.8:1 (trans-THC:cis-THC).

### Methods of Making CBDV Preparations

In the context of this application a “botanical drug substance” is an extract derived from cannabis plant material, which extract fulfils the definition of “botanical drug substance” provided in the Guidance for Industry Botanical Drug Products Draft Guidance, August 2000, US Department of Health and Human Services, Food and Drug Administration Center for Drug Evaluation and Research of: “A drug substance derived from one or more plants, algae, or macroscopic fungi. It is prepared from botanical raw materials by one or more of the following processes: pulverization, decoction, expression, aqueous extraction, ethanolic extraction, or other similar processes.”

“Plant material” is defined as a plant or plant material (e.g. bark, wood, leaves, stems, roots, flowers, fruits, seeds, berries or parts thereof) as well as exudates, and includes material falling within the definition of “botanical raw material” in the Guidance for Industry Botanical Drug Products Draft Guidance, August 2000, US Department of Health and Human Services, Food and Drug Administration Center for Drug Evaluation and Research.

The method of the invention may be used to extract cannabinoids from a specified and defined plant material known to contain such cannabinoids. Most typically, but not necessarily, the “plant material” will be “plant material” or “botanical raw material” derived from one or more cannabis plants. Most typically, but not necessarily, the one or more cannabis plants will be a specified and defined cannabis plant bred to produce a high yield of CBDV.

The term “Cannabis plant(s)” encompasses wild type Cannabis sativa and also variants thereof, including cannabis chemovars which naturally contain different amounts of the individual cannabinoids, Cannabis sativa plants which are the result of genetic crosses, self-crosses or hybrids thereof. The term “Cannabis plant material” is to be interpreted accordingly as encompassing plant material derived from one or more cannabis plants. For the avoidance of doubt, it is hereby stated that “cannabis plant material” includes dried cannabis biomass. In some embodiments, at least a portion of the cannabinoid acids in such cannabis plant material are decarboxylated.

### Cannabis Plants

The present invention utilizes cannabis plants and varieties bred to have specified, predetermined cannabinoid profiles and content. In some embodiments, a cannabinoid may be CBD, THC, CBDVA, CBDV, THCV, CBD-C1, or CBD-C4. In some embodiments, cannabis plants have specified, predetermined terpene profiles and content. In some embodiments, cannabis plants have specified, predetermined sesquiterpene profiles and content. In some embodiments, a cannabis plant is a Cannabis sativa, Cannabis indica, or Cannabis ruderalis plant.

### Cannabis Cultivation

In some embodiments, cannabis plants are propagated from cuttings taken from a mother plant. In some embodiments, a mother plant originates from a single seed source. In some embodiments, a crop is produced through asexual propagation. In some embodiments, all of the plants in a crop are all female. In some embodiments, propagation using cuttings controls genotype consistency.

In some embodiments, the growing cycle is about 12 weeks. In some embodiments, through controlled growing conditions cannabis plants take about 12 weeks to reach maturity. In some embodiments, cannabis plants are irrigated throughout their growing cycle with potable quality water. In some embodiments, no synthetic herbicides or pesticides are used in the cultivation of cannabis plants. In some embodiments, stringent hygiene conditions may be utilized to reduce ingress of pests and diseases, particularly in the absence of herbicides or pesticides. In some embodiments, control of growing conditions to reduce or eliminate environmental stresses is utilized to optimize plant material yield, cannabinoid content, and/or control disease. In some embodiments, environmental stresses may include drought, insufficient light, improper timing of light cycle, and unfavourable temperatures. In addition, regular inspection of the plants during the growing cycle allows for the detection of any rogue plants and pests. Rogue male plants may arise, though weeds should be absent due to the controlled growing conditions and media. Frequent inspections and biological control methods are used to manage any pests and diseases that may occur.

In some embodiments, through strict control of growing conditions the Cannabis plants reach maturity in approximately 12 weeks. In some embodiments, in the last weeks of growth, dense resinous flowers develop. In some embodiments, by the end of approximately week 11 the cannabinoid biosynthesis has slowed markedly, and the plants are ready for harvest.

### Cannabis Harvest and Processing

In some embodiments, the entire plant is cut and dried in a temperature and/or humidity controlled environment. In some embodiments, the temperature is about 21° C. In some embodiments, the humidity is about 38-45% RH.

In particular plants CBDV will be the principle bioactive constituent in the BDS. However, these constituents are present as the carboxylic acid CBDVA in the BRM. The acid forms slowly decarboxylate over time during drying. The leaves and flowers are stripped from the larger stems to provide the Botanical Raw Material (BRM). Under conditions of storage the loss on drying reaches equilibrium of approximately 10%. The storage conditions for the dried BRM will be dependent on the physical status of the BRM. In some embodiments, BRM is stored protected from light. In some embodiments, BRM is stored at about 15-25° C. In some embodiments, BRM is stored at about −20° C. In some embodiments, BRM is stored at about 20° C. In some embodiments, BRM is stored at about 38-42% RH.

Summary of exemplary production of a BRM:
Harvest plants
Drying (in absence of light)
Production of Botanical Raw Material (BRM) which comprises cannabinoid acids
Milling to less than 2000 μm to reduce particle size
Decarboxylation of cannabinoid acids to their neutral form (e.g. CBDVA to CBDV)

An exemplary BRM specification derived from a high CBDV variety is illustrated in Table A below:

TABLE A
Exemplary BRM specification

| Test | Method | Specification |
|---|---|---|
| Identification: | | |
| A | Visual | Complies |
| B | TLC | Corresponds to standard |
| | | (for CBDV & CBDVA) |
| C | HPLC/UV | Positive for CBDVA |
| Assay: | In-house | NLT 90% of assayed |
| CBDVA + CBDV | (HPLC/UV) | cannabinoids by peak area |
| Loss on Drying | Ph. Eur. | NMT 15% |
| Aflatoxin | UKAS method | NMT 4 ppb |
| Microbial: | | |
| TVC | Ph. Eur. | NMT10⁷ cfu/g |
| Fungi | | NMT10⁵ cfu/g |
| E. coli | | NMT10² cfu/g |
| Foreign Matter: | Ph. Eur. | NMT 2% |
| Residual Herbicides and | Ph. Eur. | Complies |
| Pesticides | | |

### Characterization of CBDV Preparations

Identification by Visual:

Macroscopic characteristics allow the features of the Cannabis plant to be distinguished from potential adulterants and substitutes. It is a visual identification against a photographic standard.

Identification by TLC:

TLC uses both retention value of the substance (Rf) and characteristic spot colour to effectively identify the variety of Cannabis. Laboratory samples are prepared for TLC analysis by extracting the dried herb. An aliquot is spotted onto a TLC plate, alongside reference samples for CBDV. Neutrals can be distinguished from the acids by comparison of the Rf value to that obtained for the standards. Identity is confirmed by comparison of Rf and colour of the sample spot, to that obtained for the appropriate standard.

Identification by HPLC:

HPLC uses retention time comparison of cannabinoids to effectively identify the variety of Cannabis. The reversed phase HPLC method is specific for CBDV and CBDVA, and therefore may be used as an identity test. Samples of biomass are extracted and centrifuged. Detection of all analytes is accomplished at 220 nm with additional confirmation of acidic analytes at 310 nm.

Assay (CBDV+CBDVA):

This assay may be used to monitor the CBDV and CBDVA content in the plant. CBDV and CBDVA assay are determined using an HPLC method. The efficiency of the decarboxylation process may be determined by dividing the % content in terms of w/w of CBDV by the total CBDV+CBDVA content.

Foreign Matter:

Foreign Matter is evaluated using the Ph.Eur. test method. Flowers, leaves and side stems are spread out in a thin layer on a clean laboratory surface. Foreign Matter is separated by hand as completely as possible and is weighed. Results are expressed as % w/w of Foreign Matter in the herbal biomass sample. Foreign Matter may comprise no more than 2% of the biomass.

Decarboxylation

THC and CBD are the principle bioactive constituents in Cannabis. However, these constituents are present as their respective carboxylic acids in Cannabis plants. In order to extract THC or CBD from cannabis plant material, it is necessary to convert the storage precursor compounds of THCA and CBDA into their more readily extractable and pharmacologically active forms. THC and CBD acids slowly decarboxylate naturally over time. The traditional way to increase rate of decarboxylation is by the application of heat. However, THCA is converted not only to THC, but also to another cannabinoid, cannabinol (CBN).

The decarboxylation procedure is generally carried out within the preparation of the starting material or botanical raw material (BRM), prior to the initiation of the extraction process.

Overview of Exemplary Extraction Process:

The BDS may be extracted from decarboxylated BRM using liquid carbon dioxide methodology. This involves continuously passing liquefied carbon dioxide through the chopped biomass, which is contained in a high-pressure vessel. The crude extract is dissolved in ethanol, cooled to a low temperature then filtered to remove precipitated constituents such as waxes. Removing ethanol and water in vacuo produces BDS containing either high concentrations of CBD or THC, depending on the biomass used.

Additional methods regarding the purification and characterization of CBD preparations are disclosed and described in EP 2 311 475, the content of which is hereby incorporated by reference in its entirety.

### Compositions and Formulations

CBDV preparations may be formulated based on the mode of intended administration. For example, in some embodiments, administration may be ocular, oral, parenteral, topical, etc. In some embodiments, a CBDV preparation may be formulated with one or more excipients to increase stability, increase shelf-life, or increase efficacy. In some embodiments, a CBDV preparation is formulated for oral administration. In some embodiments a CBDV preparation comprises sesame oil. In some embodiments a CBDV preparation comprises ethanol. In some, embodiments, the ethanol is ethanol anhydrous. In some embodiments, a CBDV preparation comprises a flavoring. In some embodiments, the flavoring may be a sweetener. In some embodiments, the sweetener may be an artificial sweetener, e.g., saccharin, acesulfame, aspartame, neotame, or sucralose. In some embodiments, the flavoring may be an artificial flavor. In some embodiments, the artificial flavor may be, e.g., vanilla, lemon, orange, lime, grapefruit, yuzu, sudachi, apple, pear, peach, grape, blueberry, strawberry, raspberry, cherry, plum, prune, raisin, cola, guarana, neroli, pineapple, apricot, banana, melon, apricot, ume, cherry, raspberry, blackberry, tropical fruit, mango, mangosteen, pomegranate, papaya, combinations thereof, or the like.

Cannabinoid preparations disclosed herein may be formulated for administration according to methods known in the art.

### Uses

Diseases, Disorders, and Conditions

CBDV preparations disclosed herein are useful in providing analgesia, neuroprotection, reduce inflammation, help alleviate nausea and emesis, as well as treat epilepsy, anxiety disorders, and glaucoma. Furthermore, CBDV preparations disclosed herein are useful in providing treatment or amelioration of symptoms in patients suffering from neurological dysfunction or the co-morbidities associated with such disorders. In some embodiments, CBDV preparations disclosed herein are more effective in treating these disorders than prior CBDV compositions. In some embodiments, a CBDV preparation of the present invention may be administered in a lower dose of CBDV than a synthetic CBDV preparation comprising the same or similar concentrations of CBDV.

Pain is a common clinical problem confronting all clinicians. Millions of people in the United States suffer from severe pain that, according to numerous recent reports, is chronically under-treated or inappropriately managed. Similarly, millions of people also suffer from severe nausea and/or frequent emesis. Moreover, all too frequently, many patients suffering from chronic, under-treated or irretraceable pain also suffer from lack of appetite, nausea and/or frequent emesis. These patients present a greater clinical challenge as they are unable to receive effective doses of oral pain medications, thereby leaving their pain unalleviated. Moreover, CBDV preparations disclosed herein can reduce a patient's nausea and vomiting, independent of any pain relief achieved. Thus, the disclosed CBDV preparations are particularly useful in patients experiencing nausea and vomiting secondary to un- or under-treated pain. In some embodiments, CBDV preparations disclosed herein are more effective in alleviating pain than prior CBDV compositions.

A notable percentage of the United States population satisfy the diagnostic criteria for alcohol use disorders (“AUDs”). The consumption of excessive amounts of alcohol results in a complex array of pharmacological effects that directly impact the ability to treat the condition. These effects directly impact the brain and include progressive neurodegeneration, impaired executive function and dependence leading to withdrawal-induced negative effects. CBDV preparations disclosed herein have neuroprotective, anxiolytic and anti-convulsant effects, which may be effective in preventing additional brain damage in persons with AUDs, while simultaneously decreasing the frequency of relapses. In some embodiments, CBDV preparations disclosed herein are more effective in treating these disorders than prior CBDV compositions.

Chronic abusers of cannabis can develop dependence and experience withdrawal symptoms when they attempt to discontinue use of the drug. Collectively cannabis dependence and withdrawal are referred to herein as cannabis use disorders. CBDV preparations disclosed herein are useful in treating cannabis use disorders. In some embodiments, CBDV preparations disclosed herein are more effective in treating these disorders than prior CBDV compositions.

Dystonia is a neurological movement disorder, with many known causes, and characterized by involuntary, continual muscular contractions causing twisting and repetitive movements or abnormal postures. In some embodiments, CBDV preparations disclosed herein are useful to reduce the muscular contractions characteristic of this disorder. CBDV preparations disclosed herein are more effective in treating these disorders than prior CBDV compositions.

The etiological pathology of many diseases relates to the inflammatory processes that are regulated by an individual's immune system. Inflammation may result from (1) an otherwise appropriate immunoresponse to an outside trauma, such as brain swelling secondary to a closed head injury; (2) an overactive immunoresponse, such as an allergic reaction or dermatitis; or (3) an inappropriate auto-immunoresponse, such as certain forms of multiple sclerosis, inflammatory bowel disorders and arthritis. Regardless of the underlying cause of the inflammation, it is therapeutically desirable under these circumstances to regulate the immune system and lessen the inflammatory response. CBDV preparations disclosed herein can regulate various steps in the immune response and could show some therapeutic benefit in the treatment of certain inflammatory diseases such as psoriatic arthritis. In some embodiments, CBDV preparations disclosed herein are more effective in treating these disorders than prior CBDV compositions.

Rheumatoid arthritis affects approximately 0.5-1% of the United States population, and autoimmune diseases in general affect more than 20 million Americans. The pain associated with rheumatoid arthritis can often be disabling. Cannabinoids have been found to be useful as an adjunct treatment for rheumatoid arthritis and joint pain secondary to other autoimmune diseases, such as inflammatory bowel disease, multiple sclerosis and systemic lupus erythematosus. In some embodiments, CBDV preparations disclosed herein are more effective in treating these disorders than prior CBDV compositions.

In addition to the above-discussed therapeutics benefits, cannabinoids, such as CBDV and CBDV prodrugs, present a variety of pharmacological benefits, including, but not limited to, anti-inflammatory, anti-convulsant, anti-psychotic, antioxidant, neuroprotective, anti-cancer and immunomodulatory effects. CBDV preparations disclosed herein are more effective in treating these disorders than prior CBDV compositions.

The present invention provides CBDV preparations and compositions and uses for treating and/or preventing any of a variety of diseases, disorders, and/or conditions, including, but not limited to those disclosed herein. In some embodiments, the present invention provides CBDV preparations and compositions and uses for treating and/or preventing diseases, disorders, or conditions associated with neurological dysfunction or neuro-differentiation. In some embodiments, diseases, disorders, or conditions associated with neurological dysfunction or neuro-differentiation are those in which neural development is defective. Such diseases, disorders or conditions are often related to the neural plasticity of the brain and can include but is not limited to seizure disorders such as epilepsy. Said seizure disorders are often associated with co-morbidities such as cognitive and psychiatric impairment which may be due to the seizures themselves or the medications used to treat the seizures. Co-morbid conditions that are known to occur in seizure disorders include but are not limited to musculoskeletal system disorders; gastrointestinal and digestive disorders; respiratory system disorders; chronic pain disorders; cerebrovascular accidents; migraine; neoplasia; arthritis/rheumatism; obesity; diabetes; infections; fractures; and allergies. Psychiatric conditions such as depression; anxiety; autism spectrum disorders; interictal dysphoric disorder; interictal behavior syndrome; and psychosis of epilepsy. Cognitive conditions such as cognitive dysfunction; language abilities; socialization; attention-deficit hyperactivity disorder; learning disability; mental retardation; and Alzheimer's disease/dementia.

In some embodiments, the disease or disorder is a seizure disorder. In some embodiments, the seizure disorder is epilepsy, Dravet syndrome, Lennox Gastaut syndrome, febrile infection related epilepsy syndrome (FIRES), Doose syndrome, Sturge Weber syndrome, CDKL5 mutation; Aicardi syndrome; bilateral polymicrogyria; Dup15q; SNAP25; benign rolandic epilepsy; juvenile myoclonic epilepsy; infantile spasm (West syndrome); and Landau-Kleffner syndrome, refractory epilepsy, juvenile spasms, West syndrome, infantile spasms, refractory infantile spasms, tuberous sclerosis complex (TSC); neurogenetic storage disorder, neuronal ceroid lipofuscinoses (NCL), Batten disease, brain abnormality, atonic, idiopathic, absence seizure, partial seizure, simple partial seizure, or complex partial seizure.

In some embodiments, the disease or disorder is a neurodegenerative disease; Alzheimer's disease; Parkinson's disease; essential tremor; amyotrophic lateral sclerosis (ALS); Huntington's disease; Friedreich's ataxia; multiple sclerosis; frontotemporal dementia; prion disease; Lewy body dementia; progressive supranuclear palsy; vascular dementia; normal pressure hydrocephalus; traumatic spinal cord injury; HIV dementia; alcohol induced neurotoxicity; Down's syndrome; movement disorders of the central and/or peripheral nervous system; motor neurone diseases (MND); spinal muscular atrophy; or any other related neurological or psychiatric neurodegenerative disease; brain damage; brain injury; brain dysfunction; dysgraphia; dysarthria; apraxia; agnosia; amnesia; dizziness; vertigo; coma; stroke; spinal cord damage; spinal cord injury; spinal cord disorders; central neuropathy; peripheral neuropathy; cranial nerve disorder; trigeminal neuralgia; tumors of the nervous system; infections of the brain or spinal cord; encephalitis; meningitis; prion disease; complex regional pain syndrome; an autonomic nervous system disorder; autonomic neuropathy; dysautonomia; postural orthostatic tachycardia syndrome (POTS); neurocardiogenic syncope (NCS); multiple system atrophy (MSA); hereditary sensory and autonomic neuropathy (HSAN); Holmes-Adie syndrome (HAS); a sleep disorder; narcolepsy; pain; migraine; cluster headache; tension headache; back pain; lower back pain; neck pain; neuropathic pain; cancer pain; allodynia; arthritic pain; inflammatory pain; a neuropsychiatric disorder; attention deficit hyperactivity disorder; autism; Tourette's Syndrome; obsessive compulsive disorder; an autism spectrum disorder; Rett syndrome; Fragile X syndrome; Angelman syndrome; hyperkinetic disorder; Tourette syndrome; dystonia; a cancer; brain cancer; glioma; breast cancer; liver cancer; lung cancer; pancreatic cancer; melanoma; ovarian cancer; gastric cancer; renal cancer; bladder cancer; addiction; nicotine addiction; smoking; alcohol addiction; drug addiction; cannabis use disorder; a mental disorder; post-traumatic stress disorder; anxiety; early psychosis; schizophrenia; a cognitive disorder; stroke; cardiac ischemia; coronary artery disease; thromboembolism; myocardial infarction; ischemic related disease; a gastrointestinal disorder; inflammatory bowel disease; Crohn's disease; ulcerative colitis; nausea; vomiting; emesis; motion sickness; chemotherapy induced nausea; chemotherapy induced nausea vomiting; inflammation; arthritis; rheumatoid arthritis; osteoarthritis; diabetes; high blood pressure; poor insulin control; appetite suppression; anorexia; neonatal hypoxic-ischemic encephalopathy (NHIE); a degenerative skeletal muscle disease; or Duchenne muscular dystrophy (DMD).

### Dosing and Administration

The exact regimen for administration of the compounds described herein may depend on the needs of the individual subject being treated, the type of treatment administered, and/or the judgment of the attending medical specialist. As used herein, the terms “subject” and “patient” includes both humans and animals. In some embodiments, the subject or patient is a human adult, human adolescent, human child, or human infant. As those skilled in the art will appreciate, the dosage administered will depend upon the condition being treated, the age, health and weight of the recipient, the type of concurrent treatment, if any, and the frequency of treatment.

In some embodiments, a CBDV preparation or a pharmaceutical composition comprising CBDV may be administered in a therapeutically effective amount. A therapeutically effective amount may be administered according to a dosing regimen comprising one or more unit doses. Generally, a therapeutically effective amount is sufficient to achieve a benefit to the subject (e.g., prophylaxis, treating, modulating, curing, preventing and/or ameliorating a disease or disorder).

A therapeutically effective amount (and/or unit dose) of a CBDV preparation or a pharmaceutical composition comprising the same for any particular patient may depend upon a variety of factors including the disease or disorder being treated; disease or disorder severity; the activity of the specific CBDV preparation or a pharmaceutical composition comprising the same employed; the specific CBDV preparation or a pharmaceutical composition comprising the same employed; the age; body weight; fitness; comorbid conditions (e.g., other than the diseases or disorder(s) being treated) general health; sex; and diet of the patient; personal history; genetic characteristic; lifestyle parameter; severity of cardiac defect and/or level of risk of cardiac defect; the time of administration; route of administration; concomitant treatments or medications; and/or rate of excretion or metabolism of the specific CBDV preparation or a pharmaceutical composition comprising the same employed; the duration of the treatment; combinations thereof; as well as other factors well known in the medical arts. In view of the present disclosure, one of ordinary skill in the art will be readily able to determine appropriate dosages depending on these and other related factors. In addition, both objective and subjective assays may optionally be employed to identify optimal dosage ranges. In some particular embodiments, appropriate doses or amounts to be administered may be extrapolated in view of the instant disclosure from dose-response curves derived from in vitro or animal model test systems.

The present invention contemplates dosing regiments comprising single as well as multiple administrations of a CBDV preparation or a pharmaceutical composition comprising the same described herein. A CBDV preparation or a pharmaceutical composition comprising the same can be administered at regular intervals, depending on the nature, severity and extent of the subject's condition. In some embodiments, a CBDV preparation or a pharmaceutical composition comprising the same may be administered periodically at regular intervals (e.g., once every year, once every six months, once every five months, once every three months, bimonthly (once every two months), monthly (once every month), biweekly (once every two weeks), weekly, daily, multiple times each day, or continuously).

A therapeutically effective amount may be administered according to a dosing regimen that may comprise multiple unit doses. For any particular CBDV preparation or a pharmaceutical composition comprising the same, a therapeutically effective amount (and/or an appropriate unit dose within an effective dosing regimen) may vary, for example, depending on route of administration, on combination with other pharmaceutical agents.

In some embodiments, a CBDV preparation or a pharmaceutical composition comprising the same described herein may be administered as a single dose. In some embodiments, a CBDV preparation or a pharmaceutical composition comprising the same described herein may be administered at regular intervals. Administration at an “interval,” as used herein, indicates that the therapeutically effective amount is administered periodically (as distinguished from a one-time dose). The interval can be determined by standard clinical techniques. In some embodiments, a CBDV preparation or a pharmaceutical composition comprising the same described herein may be administered bimonthly, monthly, twice monthly, triweekly, biweekly, weekly, twice weekly, thrice weekly, daily, twice daily, every six hours, every four hours, every two hours, or hourly. The administration interval for a given individual need not be a fixed interval, but may be varied over time, depending on the needs of the individual.

In some embodiments, a CBDV preparation or a pharmaceutical composition comprising the same described herein is administered at regular intervals indefinitely. In some embodiments, a CBDV preparation or a pharmaceutical composition comprising the same described herein is administered at regular intervals for a defined period.

It is to be further understood that for any particular subject, specific dosage regimens should be adjusted over time according to the individual need and the professional judgment of the person administering or supervising the administration of the enzyme replacement therapy and that dosage ranges set forth herein are exemplary only and are not intended to limit the scope or practice of the claimed invention.

In some embodiments, a CBDV preparation or a pharmaceutical composition comprising the same is administered as one or more doses to provide about 0.1 mg/kg/day of CBDV. In some embodiments, a CBDV preparation or a pharmaceutical composition comprising the same is administered as one or more doses to provide about 0.5 mg/kg/day of CBDV. In some embodiments, a CBDV preparation or a pharmaceutical composition comprising the same is administered as one or more doses to provide about 1 mg/kg/day of CBDV. In some embodiments, a CBDV preparation or a pharmaceutical composition comprising the same is administered as one or more doses to provide about 5 mg/kg/day of CBDV, e.g., for a 15 kg patient, 75 mg of CBDV per day would be provided. In some embodiments, a CBDV preparation or a pharmaceutical composition comprising the same is administered as one or more doses to provide about 10 mg/kg/day of CBDV. In some embodiments, a CBDV preparation or a pharmaceutical composition comprising the same is administered as one or more doses to provide about 20 mg/kg/day of CBDV. In some embodiments, a CBDV preparation or a pharmaceutical composition comprising the same is administered as one or more doses to provide about 25 mg/kg/day of CBDV. In some embodiments, a CBDV preparation or a pharmaceutical composition comprising the same is administered as one or more doses to provide about 50 mg/kg/day of CBDV. In some embodiments, a CBDV preparation or a pharmaceutical composition comprising the same is administered as one or more doses to provide about 100 mg/kg/day of CBDV.

In some embodiments, a CBDV preparation or a pharmaceutical composition comprising the same is administered in a dose of about 1 mg, about 5 mg, about 10 mg, about 20 mg, about 30 mg, about 40 mg, about 50 mg, about 60 mg, about 70 mg, about 80 mg, about 90 mg, about 100 mg, about 150 mg, about 200 mg, about 300 mg, about 400 mg, about 500 mg, about 1000 mg, about 1500 mg, or about 2000 mg of CBDV.

In some embodiments a CBDV preparation or a pharmaceutical composition comprising the same may be administered as an adjunct to conventional therapy for a disease or disorder.

### Kits

Some embodiments provide for a kit comprising a CBDV preparation or a pharmaceutical composition comprising the same and instructions for use. In some embodiments a kit further comprises a device (e.g., spray, syringe, vaporizer, inhaler, patch, etc.) for administration of said CBDV preparation or pharmaceutical composition comprising the same.

### EXAMPLES

### Example 1: Exemplary Process for Production of a Botanically Derived Purified CBDV Preparation

### Overview of the Process

The following describes the production of the botanically derived purified CBDV (>95% w/w) which has a known and constant composition which was used in the Examples below.

Plant material harvested from the Cannabis sativa L. plant was subjected to liquid carbon dioxide extraction, to produce a botanical extract containing CBDV in addition to other cannabinoids and non-cannabinoid components. The extract was then further purified by a solvent crystallization method to yield botanically derived purified CBDV. The crystallization process specifically removed other cannabinoids and plant components to yield greater than 95% (w/w) CBDV.

Both the botanical starting material and the botanical extract may be controlled by specifications.

An exemplary CBDV preparation of botanically derived purified CBDV is described in Table 1.1 below. In some embodiments, the isomeric content for each cannabinoid may also be specified.

TABLE 1.1
Specification of an exemplary botanically
derived purified CBDV preparation

| Test | Test Method | Limits |
|---|---|---|
| Appearance | Visual | Off-white/pale yellow crystals |
| Identification A | HPLC-UV | Retention time of major peak |
| | | corresponds to certified CBDV |
| | | Reference Standard |
| Identification B | GC-FID/MS | Retention time and mass spectrum |
| | | of major peak corresponds to |
| | | certified CBDV Reference Standard |
| Identification C | FT-IR | Conforms to reference spectrum for |
| | | certified CBDV Reference Standard |
| Identification D | Melting Point | 115-118° C. |
| Identification E | Specific | Conforms with certified CBDV |
| | Optical | Reference Standard; −110° to −140° |
| | Rotation | (in 95% ethanol) |
| Total Purity | Calculation | ≥95.0% |
| Chromatographic | HPLC-UV | ≥95.0% |
| Purity 1 | | |
| Chromatographic | GC-FID/MS | ≥95.0% |
| Purity 2 | | |
| CBD | HPLC-UV | NMT 4.0% w/w |
| CBD-C4 | | NMT 0.2% w/w |
| CBD-C1 | | NMT 0.15% w/w |
| CBDVA | | NMT 0.15% w/w |
| Δ⁹ THC | | NMT 0.05% w/w |
| THCV | | NMT 0.01% w/w |
| Residual | | |
| Solvents: | | |
| Alkane | GC | NMT 0.5% w/w |
| Ethanol | | NMT 0.5% w/w |
| Residual Water | Karl Fischer | NMT 1.0% w/w |

i. NMT—Not more than

The purity of the botanically derived purified CBDV preparation was greater than or equal to 95%. The botanically derived purified CBDV includes THC and other cannabinoids, e.g., CBD, CBDVA, THCV, CBD-C1, and CBD-C4.

Distinct chemotypes of the Cannabis sativa L. plant have been produced to maximize the output of the specific chemical constituents, the cannabinoids. Certain chemovars produce predominantly CBDV. Only the (−)-trans isomer of CBDV is believed to occur naturally. During purification, the stereochemistry of CBDV is not affected.

### Production of CBDV Botanical Drug Substance

An overview of the steps to produce a botanical extract, the intermediate, are as follows:

a. Growing

b. Direct drying

c. Decarboxylation

d. Extraction—using liquid CO₂

e. Winterization using ethanol

f. Filtration

g. Evaporation

High CBDV chemovars were grown, harvested, dried, baled and stored in a dry room until required. The botanical raw material (BRM) was finely chopped using an Apex mill fitted with a 1 mm screen. The milled BRM was stored in a freezer prior to extraction.

Decarboxylation of CBDVA to CBDV was carried out using heat. BRM was decarboxylated at 115° C. for 60 minutes.

Extraction was performed using liquid CO₂ to produce botanical drug substance (BDS), which was then crystalized to produce the test material. The crude CBDV BDS was winterized to refine the extract under standard conditions (2 volumes of ethanol at −20° C. for approximately 50 hours). The precipitated waxes were removed by filtration and the solvent was removed to yield the BDS.

### Production of Botanically Derived Purified CBDV Preparation

The manufacturing steps to produce the botanically derived purified CBDV preparation from BDS were as follows:

a. Crystallization using C₅-C₁₂ straight chain or branched alkane

b. Filtration

c. Vacuum drying

The BDS produced using the methodology above was dispersed in C₅-C₁₂ straight chain or branched alkane. The mixture was manually agitated to break up any lumps and the sealed container then placed in a freezer for approximately 48 hours. The crystals were isolated via vacuum filtration, washed with aliquots of cold C₅-C₁₂ straight chain or branched alkane, and dried under a vacuum of <10 mb at a temperature of 60° C. until dry. The botanically derived purified CBDV preparation was stored in a freezer at −20° C. in a pharmaceutical grade stainless steel container, with FDA food grade approved silicone seal and clamps.

### Example 2: Quantification of Composition of Synthetically Produced CBDV

A sample of synthetically produced CBDV was obtained and run on an LC-MS-IT-ToF to compare the composition of CBDV of synthetic origin with that of two samples of botanically derived purified CBDV. Both preparations comprised greater than or equal to 95% (w/w) CBDV.

As can be seen in Table 1.2 there is a difference between the CBDV of synthetic origin and that of botanically derived purified CBDV. The botanically derived purified CBDV sample comprises CBD, CBD-C1, CBDVA, CBD-C4, THC and THCV whereas the synthetically produced CBDV does not comprise these compounds. There was a very small peak detected in the spectra for synthetic CBDV which corresponded to THCV.

TABLE 1.2
Percentage of cannabinoids in botanically derived
purified CBDV preparation and synthetic CBDV

| | Botanically | Botanically | |
|---|---|---|---|
| | derived | derived | Synthetic |
| | purified CBDV (1) | purified CBDV (2) | CBDV |
| Cannabinoid | (% w/w) | (% w/w) | (% w/w) |
| CBDV | 96.0 | 99.4 | 99.9 |
| CBD | <4.0 | <4.0 | ND |
| CBD-C4 | <0.2 | <0.2 | ND |
| CBD-C1 | <0.15 | <0.15 | ND |
| CBDVA | <0.15 | <0.15 | ND |
| THC | 0.034 | 0.032 | ND |
| THCV | 0.0036 | 0.0057 | 0.0011 |

### Example 3: Autofluorescence Properties of Botanically Derived Purified CBDV and Synthetic CBDV

The physical properties of the emission and excitation spectrum of botanically derived purified CBDV and synthetic CBDV were determined in this example.

### Methods

### Test Substances

Botanically derived purified CBDV (BOT) and Synthetic CBDV (SYN) were tested in duplicates at five concentrations of 0.1 mM, 0.5 mM, 1 mM, 50 mM and 100 mM in 100% DMSO or 100% Ethanol.

### Microplate Preparation

200 μL of each sample or buffer were added into microplates in duplicate. Measurement has been performed on the Ensight multimodal plate reader (Perkin Elmer).

### Selection of Excitation Wavelength

An excitation scan was used to detect the excitation peak for each test substance. The excitation scan was set from 230 to 380 nm and below the emission wavelength of 400 nm. The excitation scan was also set from 230 to 420 nm and below the emission wavelength of 440 nm. In both cases, a step increment of 2 nm was used for these scans.

Based on these analyses, the peaks of excitation wavelengths for the two tentative emission wavelengths were determined and used to identify the optimal excitation wavelength/s.

### Selection of Emission Wavelength

In order to confirm the emission peak of the test substance, an emission scan was performed using the excitation peaks identified after the excitation scan. The excitation scan was set to start for botanically derived purified CBDV and synthetic CBDV at 20 nm above the tentative excitation values using a step increment of 2 nm. Wavelengths up to 800 nm were scanned for emission.

The wavelength giving the maximum emission for the samples was defined as the optimal emission wavelength for the chosen excitation wavelength.

### Data Presentation

Raw data were derived from the software WorkOut Plus (version 2.5, Perkin Elmer, Waltham, Mass., USA). All data extracted from the software were checked by two people for complete verification before data analysis.

These data were used to generate the data presented herein, representing in x the wavelength (nm) and in y the fluorescent intensity using WorkOut Plus software (version 2.5, Perkin Elmer).

Data were subsequently to plot these as XY graphs using GraphPad Prism (version 8.0.2, La Jolla, USA).

### Results

### Excitation Peak Points for Botanically Derived Purified CBDV and Synthetic CBDV in DMSO

Specific autofluorescence spectra distinct from the DMSO profile for each compound could only be detected at the highest concentration of 100 mM.

Representative spectroscopic data for each compound at the concentration of 100 mM are shown in FIG. 2.

Using pure DMSO as vehicle and with emission wavelength set at 400 nm, excitation peak points were identified for botanically derived purified CBDV at 282 or 326 nm. with emission wavelength set at 440 nm, excitation peak points were identified for botanically derived purified CBDV at 328 nm.

Using pure DMSO as vehicle and with emission wavelength set at 400 nm, excitation peak points were identified for synthetic CBDV at 282 or 326 nm. with emission wavelength set at 440 nm, excitation peak points were identified for botanically derived purified CBDV at 330 nm.

### Emission Peak Points for Botanically Derived Purified CBDV and Synthetic CBDV in DMSO

Specific autofluorescence for each compound could only be detected at the highest concentration of 100 mM.

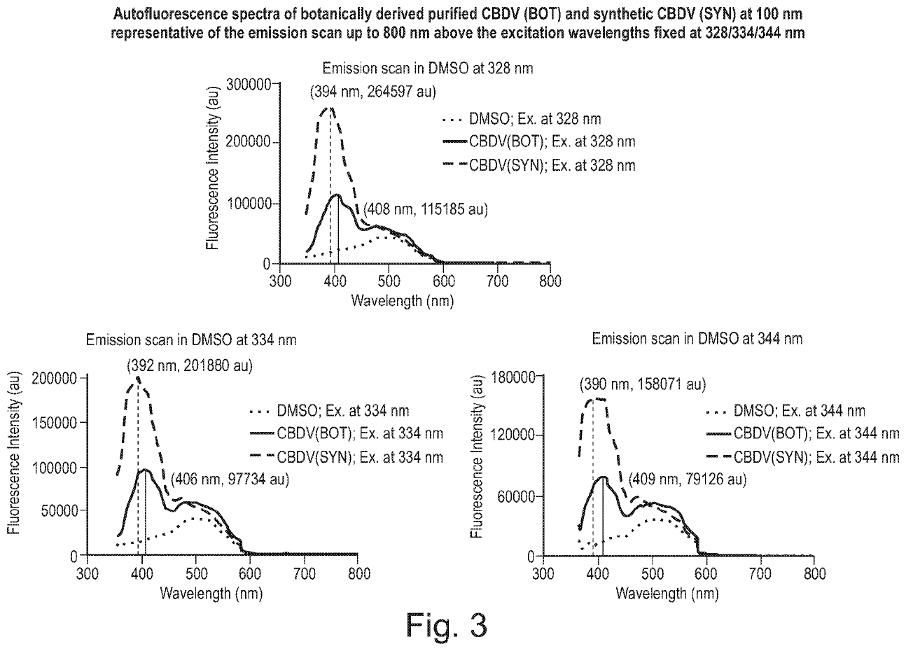

Representative spectroscopic data for each compound at the concentration of 100 mM are shown in FIG. 3.

Using DMSO as vehicle and with excitation wavelength set at 328/334/344 nm, emission peak points were identified for botanically derived purified CBDV at 408 nm, 115185 au/406 nm, 97734 au/409 nm, 79126 au; and for synthetic CBDV at 394 nm, 264597 au/392 nm, 201880 au/390 nm, 158071 au.

The monodispersed peak identified at 565 nm when excitation wavelength was set at 284 nm overlaps with that from DMSO and has not been included in the plots.

At each of the three excitation wavelengths, fluorescence intensity for CBDV(SYN) were higher than those for CBDV(BOT).

### Excitation Peak Points for Botanically Derived Purified CBDV and Synthetic CBDV in Ethanol

Specific autofluorescence spectra distinct from the ethanol profile for each compound could only be detected at the highest concentration of 100 mM.

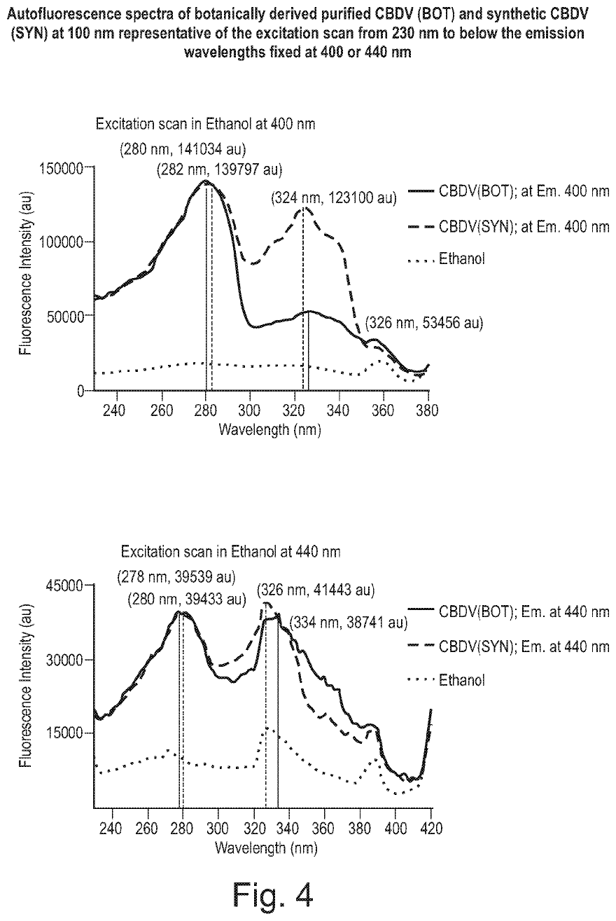

Representative spectroscopic data for each compound at the concentration of 100 mM are shown in FIG. 4.

Using ethanol as vehicle and with emission wavelength set at 400 nm, excitation peak points were identified for CBDV(BOT) at 280 nm, 141034 au and 326 nm, 53456 au and for CBDV(SYN) at 282 nm, 139797 au and 324 nm, 123100 au with peak at 324 nm at higher fluorescence intensity compared to peak from CBDV(BOT) at 326 nm.

Using ethanol as vehicle and emission wavelength set at 440 nm, excitation peak points were identified at two points for CBDV(BOT): 278 nm, 39539 au and 334 nm, 38741 au and for CBDV(SYN): 280 nm, 39433 au and 326 nm, 41443 au.

### Emission Peak Points for Botanically Derived Purified CBDV and Synthetic CBDV in Ethanol

Specific autofluorescence for each compound could only be detected at the highest concentration of 100 mM. Data suggested low specific fluorescence detection similar to that of ethanol, at all lower concentrations.

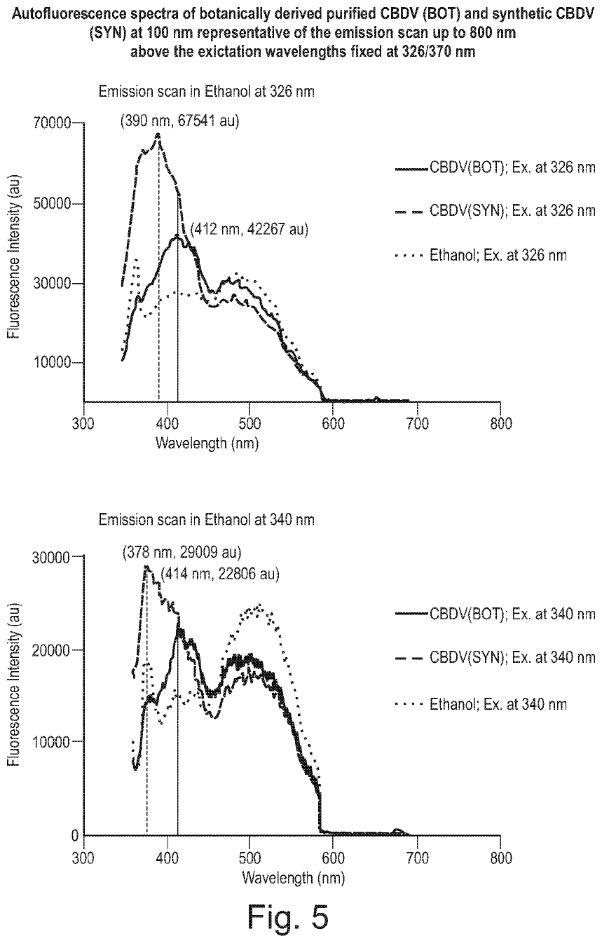

Representative spectroscopic data for each compound at the concentration of 100 mM are shown in FIG. 5.

Using ethanol as vehicle and with excitation wavelength set at 326 nm, emission peak points were identified for CBDV(BOT) at 412 nm, 42267 au and CBDV(SYN) at 390 nm, 67541 au.

Using ethanol as vehicle and with excitation wavelength set at 340 nm, excitation peak points were identified for CBDV(BOT) at 414 nm, 22806 au and CBDV(SYN) at 378 nm, 29009 au. Peaks for CBDV(SYN) have a higher fluorescence intensity.

### Conclusion

The data presented in this example suggest there is difference in the excitation and emission wavelengths of botanically derived purified CBDV and synthetic CBDV. As such there is an apparent difference in the biophysical properties of the two compounds.

### Example 4: Comparison of Botanically Derived Purified CBDV and Synthetic CBDV in an Animal Model of Schizophrenia

The effect of PCP in the novel object recognition (NOR) test is a model of visual recognition memory deficits similar to those observed in schizophrenia. The atypical antipsychotics, clozapine and risperidone, can attenuate the deficit. The study was designed to determine if botanically derived purified CBDV and/or synthetic CBDV could attenuate the deficits in novel object recognition caused by administration of PCP.

### Methods

Female hooded-Lister rats were used for this experiment. Rats were housed in groups of 5 under standard laboratory conditions under a 12 hr light: dark cycle, with lights on at 0700 hr. Testing was carried out in the light phase. Rats were randomly assigned to two treatment groups and treated with vehicle, n=15 (distilled water, i.p.) or Phencyclidine hydrochloride (PCP), n=75 (2 mg/kg, i.p. twice daily for 7-days). PCP was dissolved in distilled water. This was followed by a 7-day wash out period before the rats were tested following acute treatment with CBD, risperidone or vehicle.

Risperidone (0.1 mg/kg) was dissolved in a minimum volume of acetic acid, made up to volume with distilled water and pH adjusted to 6 with 0.1M NaOH and administered via the i.p. route in a volume of 1 ml/kg, 120 minutes prior to testing.

Botanically derived purified CBDV was tested at 2, 10 or 20 mg/kg and was dissolved in 2:1:17 (Ethanol:Cremophor:Saline 0.9%) and administered via the i.p. route in a volume of 5 ml/kg, 60 minutes prior to testing.

Synthetic CBDV at 2, 10 or 20 mg/kg was dissolved in 2:1:17 (Ethanol:Cremophor:Saline 0.9%) and administered via the i.p. route in a volume of 5 ml/kg, 60 minutes prior to testing.

Rats were allowed to habituate to the empty test box and the behavioral test room environment for one hour on day 1. Prior to behavioral testing on day 2, rats were given a further 3 minute habituation.

Following the 3 minute habituation period, the rats are given two 3 minute trials (T1 and T2) which were separated by a 1 minute inter-trial interval in the home cage during which the objects were changed. Behavior in all trials was recorded on video for subsequent blind scoring.

In the acquisition trial (T1), the animals were allowed to explore two identical objects (A1 and A2) for 3 minutes. In the retention trial (T2), the animals were allowed to explore a familiar object (A) from T1 and a novel object (B) for 3 minutes. The familiar object presented during T2 was a duplicate of objects presented in T1 in order to avoid any olfactory trails.

Object exploration was defined by animals licking, sniffing, or touching an object with the forepaws while sniffing the object, but object exploration did not include an animal leaning against, turning around, standing on or sitting on an object. The exploration time(s) of each object (A, B, familiar and novel) in each trial were recorded using two stopwatches and the following factors were calculated: total exploration time of both objects in the acquisition trial, total exploration time of both objects in the retention trial. Habituation of exploratory activity included the exploration time, as measured by the number of lines crossed, for both trials.

All data were assessed for normality using D'Agostino and Pearson normality test. Data non-normally distributed were analyzed using Kruskal-Wallis followed by planned comparisons with Dunn's correction. Normally distributed data were analyzed using one-way ANOVA followed by planned comparisons with Sidak's correction. All analyses were carried out using GraphPad Prism V7.03.

### Results

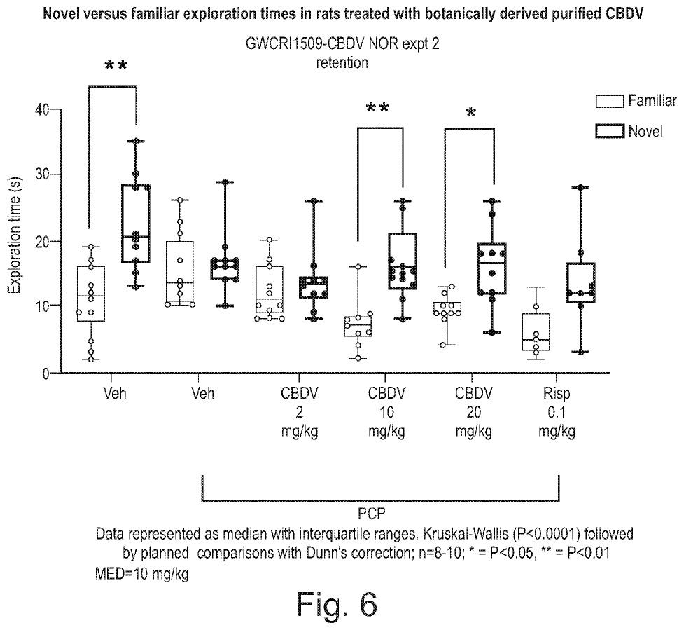

As shown in FIG. 6, botanically derived purified CBDV (2-20 mg/kg i.p., 60 min ppt) attenuated a sub-chronic PCP-induced deficit in novel object recognition in rats (n=8-10 per group), with a minimal effective dose (MED) of 10 mg/kg, (p=<0.01).

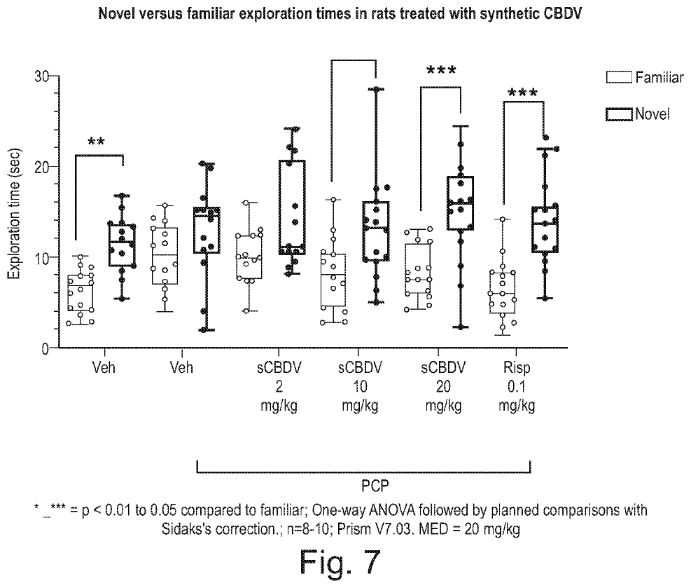

As shown in FIG. 7, synthetic CBDV (2-20 mg/kg i.p., 60 min ppt) attenuated a sub-chronic PCP-induced deficit in novel object recognition in the rat (n=8-10 per group), with a minimal effective dose (MED) of 20 mg/kg (p=<0.05).

Interestingly, the difference in potency (minimal effective dose) between the synthetic CBDV and botanically derived purified CBDV means that in a 70 kg human being dosed at 20 mg/kg/day of CBDV there is a significant difference in the amount of CBDV required. As shown below:

Rat dose of 10 mg/kg/day=(10×0.16)=1.6 mg/kg/day in a human=(1.6×70)=112 mg/day for highly purified CBDV of botanic origin.

Rat dose of 20 mg/kg/day=(20×0.16)=3.2 mg/kg/day in a human=(3.2×70)=224 mg/day for synthetic CBDV

Calculations are based on FDA dose conversion guidance from animal to human (to convert a mg/kg dose in a rat to a mg/kg human equivalent dose the rat dose is multiplied by 0.16).

### Conclusion

Botanically derived purified CBDV has been shown to be useful in attenuating the sub-chronic PCP-induced deficit in novel object recognition in rats at a much lower minimal effective dose (MED) than synthetic CBDV suggesting it would be a useful treatment option in schizophrenia and associated conditions.

Given the difference in potency, using synthetic CBDV in a human would require twice the amount of CBDV than botanically derived purified CBDV. Such a difference in potency is important given that CBDV is an expensive compound to produce whether from a synthetic or botanic route.

1. A cannabidivarin (CBDV) preparation characterized in that it comprises greater than or equal to 95% (w/w) CBDV and less than or equal to 5% (w/w) other cannabinoids, wherein the less than or equal to 5% (w/w) other cannabinoids comprise the cannabinoids tetrahydrocannabinol (THC); tetrahydrocannabivarin (THCV); cannabidiol-C1 (CBD-C1); cannabidiol (CBD); cannabidivarin acid (CBDVA) and cannabidiol-C4 (CBD-C4).

2. (canceled)

3. A method of treating a neurodevelopmental disease or condition in a subject in need thereof, comprising administering a cannabidivarin (CBDV) preparation characterized in that it comprises greater than or equal to 95% (w/w) CBDV and less than or equal to 5% (w/w) other cannabinoids, wherein the less than or equal to 5% (w/w) other cannabinoids comprise the cannabinoids tetrahydrocannabinol (THC); tetrahydrocannabivarin (THCV); cannabidiol-C1 (CBD-C1); cannabidiol (CBD); cannabidivarin acid (CBDVA) and cannabidiol-C4 (CBD-C4).

4. A method of treating epilepsy in a subject in need thereof, comprising administering a cannabidivarin (CBDV) preparation characterized in that it comprises greater than or equal to 95% (w/w) CBDV and less than or equal to 5% (w/w) other cannabinoids, wherein the less than or equal to 5% (w/w) other cannabinoids comprise the cannabinoids tetrahydrocannabinol (THC); tetrahydrocannabivarin (THCV); cannabidiol-C1 (CBD-C1); cannabidiol (CBD); cannabidivarin acid (CBDVA) and cannabidiol-C4 (CBD-4).

5. A method of treating schizophrenia in a subject in need thereof, comprising administering a cannabidivarin (CBDV) preparation characterized in that it comprises greater than or equal to 95% (w/w) CBDV and less than or equal to 5% (w/w) other cannabinoids, wherein the less than or equal to 5% (w/w) other cannabinoids comprise the cannabinoids tetrahydrocannabinol (THC); tetrahydrocannabivarin (THCV); cannabidiol-C1 (CBD-C1); cannabidiol (CBD); cannabidivarin acid (CBDVA) and cannabidiol-C4 (CBD-C4).

6. A cannabidivarin (CBDV) preparation as claimed in claim 5, wherein the preparation comprises not more than 1.5% (w/w) THC based on total amount of cannabinoid in the preparation.

7. A cannabidivarin (CBDV) preparation as claimed in claim 6, wherein the preparation comprises about 0.01% to about 0.1% (w/w) THC based on total amount of cannabinoid in the preparation.

8. A cannabidivarin (CBDV) preparation as claimed in claim 6, wherein the preparation comprises about 0.02% to about 0.05% (w/w) THC based on total amount of cannabinoid in the preparation.

9. A cannabidivarin (CBDV) preparation as claimed claim 5, wherein the preparation comprises about 0.001% to about 0.01% (w/w) THCV based on total amount of cannabinoid in the preparation.

10. A cannabidivarin (CBDV) preparation as claimed in claim 5, wherein the preparation comprises about 0.1% to about 0.25% (w/w) CBD-C1 based on total amount of cannabinoid in the preparation.

11. A cannabidivarin (CBDV) preparation as claimed in claim 5, wherein the preparation comprises about 1% to about 5% (w/w) CBD based on total amount of cannabinoid in the preparation.

12. A cannabidivarin (CBDV) preparation as claimed in claim 5, wherein the preparation comprises about 0.1% to about 0.25% (w/w) CBDVA based on total amount of cannabinoid in the preparation.

13. A cannabidivarin (CBDV) preparation as claimed in claim 5, wherein the preparation comprises about 0.05% to about 0.5% (w/w) CBD-C4 based on total amount of cannabinoid in the preparation.

14. A cannabidivarin (CBDV) preparation as claimed in claim 5, wherein at least a portion of at least one of the cannabinoids present in the CBDV preparation is isolated from cannabis plant material.

15. A cannabidivarin (CBDV) preparation as claimed in claim 5, wherein substantially all of at least one of the cannabinoids present in the CBDV preparation is isolated from cannabis plant material.

16. A cannabidivarin (CBDV) preparation as claimed in claim 5, wherein at least a portion of at least one of the cannabinoids present in the CBDV preparation is prepared synthetically.

17. A cannabidivarin (CBDV) preparation as claimed in claim 5, wherein substantially all of at least one of the cannabinoids present in the CBDV preparation is prepared synthetically.

18. A cannabidivarin (CBDV) preparation as claimed in claim 3, wherein the neurodegenerative disease or disorder is Alzheimer's disease; Parkinson's disease; essential tremor; amyotrophic lateral sclerosis (ALS); Huntington's disease; Friedreich's ataxia; multiple sclerosis; frontotemporal dementia; prion disease; Lewy body dementia; progressive supranuclear palsy; vascular dementia; normal pressure hydrocephalus; traumatic spinal cord injury; HIV dementia; alcohol induced neurotoxicity; Down's syndrome; movement disorders of the central and/or peripheral nervous system; motor neurone diseases (MND); spinal muscular atrophy; or any other related neurological or psychiatric neurodegenerative disease; brain damage; brain injury; brain dysfunction; dysgraphia; dysarthria; apraxia; agnosia; amnesia; dizziness; vertigo; coma; stroke; spinal cord damage; spinal cord injury; spinal cord disorders; central neuropathy; peripheral neuropathy; cranial nerve disorder; trigeminal neuralgia; tumors of the nervous system; infections of the brain or spinal cord; encephalitis; meningitis; prion disease; complex regional pain syndrome; an autonomic nervous system disorder; autonomic neuropathy; dysautonomia; postural orthostatic tachycardia syndrome (POTS); neurocardiogenic syncope (NCS); multiple system atrophy (MSA); hereditary sensory and autonomic neuropathy (HSAN); Holmes-Adie syndrome (HAS); a sleep disorder; narcolepsy; pain; migraine; cluster headache; tension headache; back pain; lower back pain; neck pain; neuropathic pain; cancer pain; allodynia; arthritic pain; inflammatory pain; a neuropsychiatric disorder; attention deficit hyperactivity disorder; autism; Tourette's Syndrome; obsessive compulsive disorder; an autism spectrum disorder; Rett syndrome; Fragile X syndrome; Angelman syndrome; hyperkinetic disorder; mitochondrial disease; dystonia; a cancer; brain cancer; glioma; breast cancer; liver cancer; lung cancer; pancreatic cancer; melanoma; ovarian cancer; gastric cancer; renal cancer; bladder cancer; addiction; nicotine addiction; smoking; alcohol addiction; drug addiction; cannabis use disorder; a mental disorder; post-traumatic stress disorder; anxiety; early psychosis; schizophrenia; a cognitive disorder; stroke; cardiac ischemia; coronary artery disease; thromboembolism; myocardial infarction; ischemic related disease; a gastrointestinal disorder; inflammatory bowel disease; Crohn's disease; ulcerative colitis; nausea; vomiting; emesis; motion sickness; chemotherapy induced nausea; chemotherapy induced nausea vomiting; inflammation; arthritis; rheumatoid arthritis; osteoarthritis; diabetes; high blood pressure; poor insulin control; appetite suppression; anorexia; neonatal hypoxic-ischemic encephalopathy (NHIE); a degenerative skeletal muscle disease; or Duchenne muscular dystrophy (DMD).

19. A cannabidivarin (CBDV) preparation as claimed in claim 4, wherein the epilepsy is Dravet syndrome, Lennox Gastaut syndrome, febrile infection related epilepsy syndrome (FIRES), Doose syndrome, Sturge Weber syndrome, CDKL5 mutation; Aicardi syndrome; bilateral polymicrogyria; Dup15q; SNAP25; benign rolandic epilepsy; juvenile myoclonic epilepsy; infantile spasm (West syndrome); and Landau-Kleffner syndrome, refractory epilepsy, juvenile spasms, West syndrome, infantile spasms, refractory infantile spasms, tuberous sclerosis complex (TSC); neurogenetic storage disorder, neuronal ceroid lipofuscinoses (NCL), Batten disease, brain abnormality, atonic, idiopathic, absence seizure, partial seizure, simple partial seizure, or complex partial seizure.
